(19) United States
(12) Patent Application Publication
(10) Pub. No.: US 2019/0361006 A1
(43) Pub. Date: Nov. 28, 2019

(54) METHOD OF PREDICTING CLINICAL OUTCOME OF ANTICANCER AGENTS

(71) Applicant: MITRA RXDX, INC., Woburn, MA (US)

(72) Inventors: Aaron GOLDMAN, Woburn, MA (US); Pradip K. MAJUMDER, Woburn, MA (US); Padhma D. RADHAKRISHNAN, Woburn, MA (US)

(21) Appl. No.: 16/484,391

(22) PCT Filed: Feb. 7, 2018
(86) PCT No.: PCT/US2018/017297

§ 371 Date: Aug. 7, 2019

Related U.S. Application Data
(60) Provisional application No. 62/456,550, filed on Feb. 8, 2017.
(60) Provisional application No. 62/464,993, filed on Feb. 28, 2017.
(60) Provisional application No. 62/596,060, filed on Dec. 7, 2017.

(51) Int. Cl. G01N 33/50; C12N 5/09; G16C 20/30

(52) U.S. Cl. CPC G01N 33/5011; G01N 2800/52; G16C 20/30; C12N 5/0694

(57) ABSTRACT

The invention provides methods of predicting responsiveness of an individual having a cancer to administration of an anticancer drug regimen using a tumor tissue culture capable of mimicking physiologically relevant signaling, where the prediction depends in part on an immune contexture phenotype in the tumor tissue culture.

### CROSS-REFERENCE TO RELATED APPLICATIONS

This application claims priority from U.S. Provisional Patent Application No. 62/456,550, filed Feb. 8, 2017; U.S. Provisional Patent Application No. 62/464,993, filed Feb. 28, 2017; and U.S. Provisional Patent Application No. 62/596,060, filed Dec. 7, 2017, the contents of each of which are incorporated herein by reference in its entirety.

### TECHNICAL FIELD

This application pertains to prognostic and therapeutic methods involving determining the responsiveness of an individual having cancer to one or more therapeutic agents based on a clinical response predictor.

### BACKGROUND

Cultured tumor tissue explants derived from patients have been used to predict responsiveness to administration of anticancer therapies in efforts to select appropriate drug treatment regimens for a given patient. However, predictions based on such tumor tissue cultures are prone to yielding false positives and false negatives. The selection of both the tumor tissue culture conditions and the combination of assays carried out on the tumor tissue cultures plays an important role in the accuracy and sensitivity of methods of predicting responsiveness to anticancer therapies based on these cultures. There is an unmet need to improve and refine methods of assessing responsiveness to anticancer therapies using patient-derived tumor tissue cultures.

### SUMMARY

In some embodiments, there is provided a method of predicting responsiveness of an individual having a cancer to administration of an anticancer drug regimen, the method comprising: a) obtaining a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell; b) inputting the readout into a predictive model; c) using the predictive model to generate an output; and d) using the output to predict responsiveness of the individual to administration of the anticancer drug regimen.

In some embodiments, there is provided a method of classifying likely responsiveness of an individual having a cancer to administration of an anticancer drug regimen, comprising: a) obtaining a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell; b) inputting the readout into a predictive model; c) using the predictive model to generate an output; and d) using the output to classify the likely responsiveness of the individual to administration of the anticancer drug regimen.

In some embodiments, there is provided a computer-implemented method for predicting responsiveness of an individual having a cancer to administration of an anticancer drug regimen, the method comprising: a) accessing a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell; b) inputting the readout into a predictive model; c) using the predictive model to generate an output; and d) using the output to predict responsiveness of the individual to administration of the anticancer drug regimen.

In some embodiments, according to any of the methods described above, the predictive model comprises an algorithm that uses each of the assessment scores as input and generates the output. In some embodiments, the algorithm comprises multiplying each of the input assessment scores with a corresponding weightage coefficient to obtain a plurality of weighted assessment scores; and combining the plurality of weighted assessment scores to generate the output.

In some embodiments, according to any of the methods described above, the predictive model comprises a first algorithm that uses each of the assessment scores for the first set of a plurality of assays as input and generates a preliminary output, and a second algorithm that uses the preliminary output and each of the assessment scores for the second set of one or more assays as input and generates the output. In some embodiments, the first algorithm comprises multiplying each of the input assessment scores with a corresponding weightage coefficient to obtain a plurality of weighted assessment scores; and combining the plurality of weighted assessment scores to generate the preliminary output. In some embodiments, the preliminary output predicts a primary degree of responsiveness of the individual to administration of the anticancer drug regimen, and wherein the second algorithm comprises adjusting the primary predicted degree of responsiveness based on the input assessment scores to generate the output. In some embodiments, the second set of one or more assays consists of the assay for tumor infiltration of an immune cell, and adjusting the primary predicted degree of responsiveness comprises predicting a secondary degree of responsiveness of the individual to administration of the anticancer drug regimen based on the input assessment score, and 1) adjusting the primary predicted degree of responsiveness by decreasing the predicted degree of responsiveness if the secondary predicted degree of responsiveness is lower than the primary predicted degree of responsiveness and the input assessment score is below a first threshold, thereby generating the output; or 2) adjusting the primary predicted degree of responsiveness by increasing the predicted degree of responsiveness if the secondary predicted degree of responsiveness is greater than the primary predicted degree of responsiveness and the input assessment score is above a second threshold, thereby generating the output.

In some embodiments, according to any of the methods described above, the output predicts complete clinical response, partial clinical response, or no clinical response of the individual to administration of the anticancer drug regimen.

In some embodiments, according to any of the methods described above, the output predicts response or no response of the individual to administration of the anticancer drug regimen.

In some embodiments, according to any of the methods described above, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture.

In some embodiments, according to any of the methods described above, the immune cell is an NK cell.

In some embodiments, according to any of the methods described above, the first set of a plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof.

In some embodiments, according to any of the methods described above, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual.

In some embodiments, according to any of the methods described above, step a) further comprises conducting the plurality of assays on the tumor tissue culture and/or step a) further comprises preparing the tumor tissue culture by culturing tumor tissue from the individual on the tumor microenvironment platform.

In some embodiments, according to any of the methods described above, the assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step a) further comprises conducting the plurality of assays on the reference tumor tissue culture; and/or step a) further comprises preparing the reference tumor tissue culture by culturing tumor tissue from the individual on the tumor microenvironment platform.

In some embodiments, there is provided a method of treating cancer in an individual in need thereof, the method comprising administering to the individual an anticancer drug regimen to which the individual is predicted to respond according to any of the methods described above. In some embodiments, the individual is predicted to have a complete clinical response or partial clinical response to administration of the anticancer drug regimen.

In some embodiments, according to any of the methods described above, the anticancer drug regimen comprises an anticancer agent and/or an immunotherapeutic agent. In some embodiments, the anticancer drug regimen comprises an anticancer agent. In some embodiments, the anticancer drug regimen comprises an immunotherapeutic agent. In some embodiments, the anticancer drug regimen comprises an anticancer agent and an immunotherapeutic agent. In some embodiments, the anticancer agent is selected from the group consisting of adriamycin, gemcitabine, palbociclib, docetaxel, fulvestrant, carboplatin, exemestane, everolimus, vinorelbine, olaparib, capecitabine, cyclophosphamide, methotrexate, fluorouracil, and any combination thereof. In some embodiments, the immunotherapeutic agent is an immune checkpoint inhibitor. In some embodiments, the immunotherapeutic agent is selected from the group consisting of nivolumab, ipilimumab, pembrolizumab, atezolizumab, and any combination thereof.

In some embodiments, there is provided a method of predicting responsiveness to a therapeutic agent for treating cancer in an individual in need thereof, the method comprising: a) obtaining a readout comprising assessment scores from a plurality of assays conducted on a tumor tissue culture, wherein the tumor tissue culture comprises i) a tumor microenvironment platform cultured with tumor tissue from the individual; and ii) the therapeutic agent; b) converting the readout into a sensitivity index; and c) using the sensitivity index to predict responsiveness to the therapeutic agent, wherein at least one of the plurality of assays does not relate to a tumor cell phenotype.

In some embodiments, according to any of the methods described above, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, immune cell contexture assays, and any combination thereof. In some embodiments, at least one of the plurality of assays comprises quantifying activity and/or infiltration of one or more immune cell in the tumor tissue. In some embodiments, at least one of the plurality of assays comprises quantifying activity and/or infiltration of T cells in the tumor tissue. In some embodiments, the T cells are cytotoxic T cells. In some embodiments, at least one of the plurality of assays comprises quantifying activity and/or infiltration of NK cells in the tumor tissue. In some embodiments, at least one of the plurality of assays comprises quantifying the expression of one or more cytokines in the tumor tissue culture.

In some embodiments, according to any of the methods described above, the tumor microenvironment platform comprises an extracellular matrix composition comprising culture medium and one or more of collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, or autologous peripheral blood nuclear cells (PBNC).

In some embodiments, according to any of the methods described above, step a) further comprises culturing tumor tissue obtained from the individual with the tumor microenvironment platform and adding the therapeutic agent to the tumor microenvironment platform. In some embodiments, step a) further comprises conducting the plurality of assays on the tumor tissue culture to generate assessment scores, thereby producing the readout. In some embodiments, step b) further comprises multiplying the assessment score of each of the plurality of assays with a weightage score for the assay to obtain a weighted assay score for each of the plurality of assays; and combining the weighted assay scores for each of the plurality of assays to obtain the sensitivity index.

In some embodiments, according to any of the methods described above, the sensitivity index predicts complete clinical response, partial clinical response, or no clinical response to the therapeutic agent in the individual.

In some embodiments, according to any of the methods described above, the therapeutic agent is a chemotherapeutic agent or an immunotherapeutic agent.

In some embodiments, according to any of the methods described above, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, immune contexture assays, and any combination thereof.

In some embodiments, there is provided a method of treating cancer in an individual in need thereof, the method comprising administering to the individual a therapeutic agent having a sensitivity index according to any of the methods described above that predicts responsiveness.

In some embodiments, according to any of the method of treating cancer described above, the therapeutic agent has a sensitivity index that predicts complete clinical response or partial clinical response in the individual.

In some embodiments, according to any of the methods described above, the therapeutic agent is an immune checkpoint inhibitor. In some embodiments, the immune checkpoint inhibitor is an antagonistic antibody targeting an immune checkpoint molecule. In some embodiments, the immune checkpoint inhibitor is pembrolizumab or nivolumab.

In some embodiments, according to any of the methods described above, the plurality of therapeutic agents comprises a plurality of immune checkpoint inhibitors. In some embodiments, the plurality of immune checkpoint inhibitors comprises a plurality of antagonistic antibodies targeting an immune checkpoint molecule. In some embodiments, the plurality of immune checkpoint inhibitors comprises pembrolizumab and nivolumab.

In some embodiments, according to any of the methods described above, the individual is human.

### BRIEF DESCRIPTION OF THE DRAWINGS

FIG. 1 shows results for H&E staining and IHC analysis for cleaved caspase 3, MICA/B, and CD56 expression in tumor tissue cultured in the tumor microenvironment platform treated with gefitinib, osimertinib+Pembrolizumab, or vehicle control for 3 days (T3).

FIGS. 2A-2C show NK cell spatial heterogeneity in tissue cultured in the tumor microenvironment platform treated with various conventional and immuno-modulatory therapies. FIG. 2A shows IHC analysis for NK cell marker CD56 under treatment and control conditions. Areas of tumor cells (T), normal stroma (S), and normal cells (N) are indicated. FIG. 2B shows pairwise quantitation of the ratio of CD56+ cells in areas of tumor cells vs areas of stroma in untreated (Vehicle) and drug pressure (Rx) conditions. FIG. 2C shows the fold change in the CD56+ tumor:stroma ratio from untreated to drug pressure conditions.

FIGS. 3A and 3B show changes in NK cell spatial heterogeneity in tissue cultured in the tumor microenvironment platform under pressure from treatment with immunotherapy-containing drug regimens (Rx) nivolumab+adriamycin (FIG. 3A) and gemcitabine+nivolumab+ipilimumab (FIG. 3B) as compared to vehicle control (Vehicle). Areas of tumor cells (T) and normal stroma (S) are indicated.

FIG. 4 shows changes in NK cell spatial heterogeneity in tissue cultured in the tumor microenvironment platform under pressure from treatment with anticancer drug regimens alpelisib+fulvestrant and everolimus+fulvestrant as compared to vehicle control. Areas of normal stroma are indicated by the bounded regions.

FIG. 5 shows changes in NK cell spatial heterogeneity in tissue cultured in the tumor microenvironment platform under pressure from treatment with anticancer drug regimens i) trametinib+everolimus+cetuximab, ii) pembrolizumab+capecitabine, iii) 5-FU+mitomycin C+temezolomide, and iv) trametinib+cetuximab+capecitabine as compared to vehicle control. Areas of tumor are indicated by the bounded regions.

FIGS. 6A and 6B show changes in pro-inflammatory cytokine (FIG. 6A) and anti-inflammatory cytokine (FIG. 6B) expression in tumor tissue from HER2−/ER+/PR+ breast cancer patients cultured in the tumor microenvironment platform from control (Vehicle) to various treatment (Rx) conditions. Treatments included palbociclib, pembrolizumab, and docetaxel.

### DETAILED DESCRIPTION

The present invention is based at least in part on the observation that a tumor tissue culture as described herein, optionally combined with a machine learning strategy, can more accurately predict responsiveness of an individual with a cancer to administration of an anticancer drug regimen when the prediction is based in part on a measure of certain markers in response to administration of the anticancer drug regimen, e.g., tumor infiltration of an immune cell. Specific phenotypic markers, including an immune cell (e.g., NK cell) tumor infiltration marker, induced under therapy pressure may be used to provide a quantitative measure of clinical outcome, for example, when being appropriately weighted by a machine learning algorithm. Accordingly, the present invention provides compositions, kits, articles of manufacture, and methods for predicting responsiveness of an individual having a cancer to administration of an anticancer drug regimen, such as an anticancer drug regimen comprising an anticancer agent and/or an immunotherapeutic agent. Also provided are methods of treating cancer utilizing such predictive methods.

We have previously established and optimized a tumor microenvironment platform for culturing tumor tissue explants that mimics the native human tumor environment (see US Patent No. 2014/0228246, incorporated herein in its entirety). While this live tumor assay had been shown to accurately predict the antitumor effects of a number of different therapies using a variety of tumor phenotypic markers, the inclusion of immune contexture phenotypic markers, such as markers for tumor infiltration of an immune cell, were found to improve the predictive accuracy of the live tumor assay. The present invention describes the use of a live tissue assay, which in some cases harnesses a multi-dimensional phenotypic “reflex” and optionally a machine learning algorithm, to predict the clinical outcome of cancer therapy drugs, such as immune modulatory drugs, in a single individual.

In some embodiments, the live tissue assay comprises a tumor tissue derived from an individual, an ECM composition, and optionally serum, plasma, peripheral blood nuclear cells (PBNCs), and/or granulocytes (such as autologous serum, plasma, PBNCs, and/or granulocytes). In some embodiments, the live tissue assay mimics aspects of the immune complex and compartment of the native tumor environment. Existing technologies (e.g., foundation medicine, in vitro diagnostics, and quantitative pathology such as Immuno Score) rely on a priori knowledge of the tumor-immune contexture. For example, others have illustrated that the level of infiltration of tumor lymphocytes can predict, prior to treatment, whether the patient is likely to respond to a given therapy. The present invention demonstrates that in some cases, infiltration of lymphocytes into a live tumor tissue culture can be induced under therapy pressure, and the amount of infiltration can be used in predicting antitumor effects such as diminished proliferation and increased cell death of tumor cells.

It is contemplated that in some embodiments, the live tumor tissue assay, making use of certain phenotypic markers, such as tumor infiltration of an immune cell, in addition to the previously described tumor-associated markers, can accurately predict the clinical efficacy of a wide array of cancer therapeutic agents, including immunomodulatory agents. It is also contemplated that in some embodiments, the invention can further predict the clinical efficacy of alternative immune modulatory therapeutics such as antitumor vaccines, chimeric antigen receptor T-cells (CAR-T), cytokine invigoration or even viral/bacterial immune stimulation strategies, and can be applicable to many different drugs and regimens including combination therapies.

### Definitions

Unless defined otherwise, the meanings of all technical and scientific terms used herein are those commonly understood by one of skill in the art to which this invention belongs. One of skill in the art will also appreciate that any methods and materials similar or equivalent to those described herein can also be used to practice or test the invention.

For use herein, unless clearly indicated otherwise, use of the terms “a”, “an,” and the like refers to one or more.

In this application, the use of “or” means “and/or” unless expressly stated or understood by one skilled in the art. In the context of a multiple dependent claim, the use of “or” refers back to more than one preceding independent or dependent claim.

Reference to “about” a value or parameter herein includes (and describes) embodiments that are directed to that value or parameter per se. For example, description referring to “about X” includes description of “X.”

It is understood that aspect and embodiments of the invention described herein include “comprising,” “consisting,” and “consisting essentially of” aspects and embodiments.

### Methods

### Predicting Responsiveness

In some embodiments, there is provided a method of predicting responsiveness of an individual having a cancer to administration of an anticancer drug regimen, the method comprising: a) obtaining a readout comprising an assessment score for each of a plurality of assays conducted on a tumor tissue culture treated with the anticancer drug regimen, wherein the tumor tissue culture comprises a tumor tissue from the individual cultured on a tumor microenvironment platform; b) converting the readout into a sensitivity index; and c) using the sensitivity index to predict responsiveness to the anticancer drug regimen, wherein at least one of the plurality of assays is an assay for tumor infiltration of an immune cell. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell. In some embodiments, the tumor microenvironment platform comprises an extracellular matrix composition comprising one or more of (such as at least 3, 4, 5, or more of) basement membrane proteins, cytoskeletal proteins, and matrix proteins In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and immune cell contexture assays. In some embodiments, the assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the anticancer drug regimen comprises an anticancer agent and/or an immunotherapeutic agent. In some embodiments, the anticancer agent includes a cytostatic or cytotoxic agent. In some embodiments, the anticancer agent includes a targeted anticancer agent, such as a targeted antibody or targeted small molecule (e.g., protein inhibitor, such as kinase inhibitor). In some embodiments, the anticancer agent includes adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the immunotherapeutic agent includes an immunomodulatory agent, e.g., an immune checkpoint inhibitor or an immunostimulatory agent. In some embodiments, the immunotherapeutic agent includes nivolumab, ipilimumab, pembrolizumab, atezolizumab, and any combination thereof. In some embodiments, step a) further comprises conducting the plurality of assays on the tumor tissue culture and/or step a) further comprises preparing the tumor tissue culture by culturing tumor tissue from the individual on the tumor microenvironment platform. In some embodiments, a) further comprises conducting the plurality of assays on the reference tumor tissue culture; and/or step a) further comprises preparing the reference tumor tissue culture by culturing tumor tissue from the individual on the tumor microenvironment platform.

As used herein, a “readout” refers to a set of one or more assessment scores.

In some embodiments, according to any of the methods described herein employing a tumor microenvironment platform, the tumor microenvironment platform comprises an extracellular matrix composition. In some embodiments, the extracellular matrix composition comprises at least 2 (such as at least 3, 4, 5, or more) of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the extracellular matrix composition comprises no more than 6 (such as no more than 5, 4, 3, or fewer) of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the extracellular matrix composition comprises at least 2 (such as at least 3, 4, 5, or more) proteins selected from basement membrane proteins, cytoskeletal proteins, and matrix proteins. In some embodiments, the extracellular matrix composition comprises no more than 6 (such as no more than 5, 4, 3, or fewer) proteins selected from basement membrane proteins, cytoskeletal proteins, and matrix proteins. In some embodiments, the tumor microenvironment platform further comprises serum, plasma, and/or PBNCs. In some embodiments, at least one of the serum, plasma, and/or PBNCs are autologous to the individual. In some embodiments, at least one of the serum, plasma, and/or PBNCs are heterologous to the individual. In some embodiments, the PBNCs are peripheral blood mononuclear cells (PBMCs).

Thus, in some embodiments, according to any of the methods described herein employing a tumor microenvironment platform, the tumor microenvironment platform comprises a) an extracellular matrix composition comprising at least 2 (such as at least 3, 4, 5, or more) of collagen 1, collagen 3, collagen 4, collagen 6, Fibronectin, Vitronectin, Cadherin, Filamin A, Vimentin, Osteopontin, Laminin, Decorin, and Tenascin C; and b) serum, plasma, and/or PBNCs. In some embodiments, the extracellular matrix composition comprises no more than 6 (such as no more than 5, 4, 3, or fewer) of collagen 1, collagen 3, collagen 4, collagen 6, Fibronectin, Vitronectin, Cadherin, Filamin A, Vimentin, Osteopontin, Laminin, Decorin, and Tenascin C. In some embodiments, at least one of the serum, plasma, and/or PBNCs are autologous to the individual. In some embodiments, at least one of the serum, plasma, and/or PBNCs are heterologous to the individual. In some embodiments, the PBNCs are peripheral blood mononuclear cells (PBMCs).

In some embodiments, according to any of the methods described herein employing a tumor microenvironment platform, the tumor microenvironment platform comprises a) an extracellular matrix composition comprising at least 2 (such as at least 3, 4, 5, or more) proteins selected from basement membrane proteins, cytoskeletal proteins, and matrix proteins; and b) serum, plasma, and/or PBNCs. In some embodiments, the extracellular matrix composition comprises no more than 6 (such as no more than 5, 4, 3, or fewer) proteins selected from basement membrane proteins, cytoskeletal proteins, and matrix proteins. In some embodiments, at least one of the serum, plasma, and/or PBNCs are autologous to the individual. In some embodiments, at least one of the serum, plasma, and/or PBNCs are heterologous to the individual. In some embodiments, the PBNCs are peripheral blood mononuclear cells (PBMCs).

In some embodiments, according to any of the methods described herein employing an anticancer drug regimen, the anticancer drug regimen comprises one or more anticancer agents and/or one or more immunotherapeutic agents. In some embodiments, the anticancer drug regimen comprises one or more anticancer agents. In some embodiments, the anticancer drug regimen comprises one or more immunotherapeutic agents. In some embodiments, the anticancer drug regimen comprises one or more anticancer agents and one or more immunotherapeutic agents. In some embodiments, the one or more anticancer agents include a cytostatic or cytotoxic agent. In some embodiments, the one or more anticancer agents include a targeted anticancer agent, such as a targeted antibody or targeted small molecule (e.g., protein inhibitor, such as kinase inhibitor). In some embodiments, the one or more anticancer agents include adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the one or more immunotherapeutic agents include an immunomodulatory agent, e.g., an immune checkpoint inhibitor or an immunostimulatory agent. In some embodiments, the one or more immunotherapeutic agents include nivolumab, ipilimumab, pembrolizumab, atezolizumab, and any combination thereof.

In some embodiments, according to any of the methods described herein employing an assay for tumor infiltration of an immune cell, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell.

In some embodiments, according to any of the methods described herein employing an assessment score for an assay, the assessment score is generated based on a comparison between i) the result of the assay conducted on the tumor tissue culture treated with the anticancer drug regimen; and ii) the result of the assay conducted on a reference tumor tissue culture, wherein the reference tumor tissue culture comprises a tumor tissue from the individual cultured on the tumor microenvironment platform. In some embodiments, the assessment score is generated, for example, by taking the ratio of i) a numeric quantification of the result of the assay conducted on the tumor tissue culture treated with the anticancer drug regimen to ii) the numeric quantification of the result of the assay conducted on the reference tumor tissue culture. In some embodiments, the reference tumor tissue culture is not treated with the anticancer drug regimen.

In some embodiments, according to any of the methods described herein employing a tumor tissue culture from an individual, the method comprises culturing a tumor tissue from the individual on a tumor microenvironment platform as described herein to produce the tumor tissue culture.

In some embodiments, according to any of the methods described herein employing a plurality of assays conducted on a tumor tissue culture, the method comprises conducting the plurality of assays on the tumor tissue culture.

In some embodiments, there is provided a method of predicting responsiveness of an individual having a cancer to administration of an anticancer drug regimen, the method comprising: a) conducting a plurality of assays on a tumor tissue culture treated with the anticancer drug regimen, wherein the tumor tissue culture comprises a tumor tissue from the individual cultured on a tumor microenvironment platform, and obtaining a readout comprising an assessment score for each of the plurality of assays; b) converting the readout into a sensitivity index; and c) using the sensitivity index to predict responsiveness to the anticancer drug regimen, wherein at least one of the plurality of assays is an assay for tumor infiltration of an immune cell. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and immune cell contexture assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the anticancer drug regimen comprises an anticancer agent and/or an immunotherapeutic agent. In some embodiments, the anticancer agent includes a chemotherapeutic agent, such as a cytostatic or cytotoxic agent. In some embodiments, the anticancer agent is a targeted therapeutic agent, such as a targeted antibody or targeted small molecule (e.g., protein inhibitor, such as kinase inhibitor). In some embodiments, the immunotherapeutic agent includes an immunomodulatory agent, e.g., an immune checkpoint inhibitor or an immunostimulatory agent.

In some embodiments, there is provided a method of predicting responsiveness of an individual having a cancer to administration of an anticancer drug regimen, the method comprising: a) preparing a tumor tissue culture by culturing a tumor tissue from the individual on a tumor microenvironment platform; b) conducting a plurality of assays on the tumor tissue culture that has been treated with the anticancer drug regimen and obtaining a readout comprising an assessment score for each of the plurality of assays; c) converting the readout into a sensitivity index; and d) using the sensitivity index to predict responsiveness to the anticancer drug regimen, wherein at least one of the plurality of assays does not relate to a tumor cell phenotype. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and immune cell contexture assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the anticancer drug regimen comprises an anticancer agent and/or an immunotherapeutic agent. In some embodiments, the anticancer agent includes a chemotherapeutic agent, such as a cytostatic or cytotoxic agent. In some embodiments, the anticancer agent is a targeted therapeutic agent, such as a targeted antibody or targeted small molecule (e.g., protein inhibitor, such as kinase inhibitor). In some embodiments, the immunotherapeutic agent includes an immunomodulatory agent, e.g., an immune checkpoint inhibitor or an immunostimulatory agent.

In some embodiments, there is provided a method of predicting responsiveness of an individual having a cancer to administration of an anticancer drug regimen, the method comprising: a) obtaining a readout comprising an assessment score for each of a plurality of assays conducted on a tumor tissue culture treated with the anticancer drug regimen, wherein the tumor tissue culture comprises a tumor tissue from the individual cultured on a tumor microenvironment platform; b) converting the readout into a sensitivity index; c) determining the ratio of an immune cell (e.g., NK cells) in a region of tumor cells versus a region of normal stroma in the tumor tissue culture, thereby generating a tumor:stroma immune cell ratio for the treated tumor tissue culture; and d) using the sensitivity index and the tumor:stroma immune cell ratio to predict responsiveness to the anticancer drug regimen. In some embodiments, the immune cell is an NK cell. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises one or more of serum, plasma, and PBNCs. In some embodiments, at least one of the serum, plasma, and PBNCs are autologous to the individual. In some embodiments, at least one of the serum, plasma, and PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and immune cell contexture assays. In some embodiments, the assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step c) further comprises determining the ratio of the immune cell in a region of tumor cells versus a region of normal stroma in the reference tumor tissue culture, thereby generating a tumor:stroma immune cell ratio for the reference tumor tissue culture. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values. In some embodiments, using the sensitivity index and the tumor:stroma immune cell ratio to predict responsiveness comprises predicting the responsiveness to be the degree of responsiveness associated with the range of values in which the sensitivity index lies if the treated tumor:stroma immune cell ratio does not decrease compared to the reference tumor:stroma immune cell ratio. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, using the sensitivity index and the tumor:stroma immune cell ratio to predict responsiveness comprises predicting the responsiveness to be a lesser degree of responsiveness than that associated with the range of values in which the sensitivity index lies if the treated tumor:stroma immune cell ratio for the treated tissue culture decreases (such as decreases by a first threshold) compared to the reference tumor:stroma immune cell ratio. In some embodiments, if the treated tumor:stroma immune cell ratio decreases (such as decreases by a second threshold) compared to the reference tumor:stroma immune cell ratio, the responsiveness is predicted to be no clinical response. In some embodiments, using the sensitivity index and the tumor:stroma immune cell ratio to predict responsiveness comprises predicting the responsiveness to be a greater degree of responsiveness than that associated with the range of values in which the sensitivity index lies if the treated tumor:stroma immune cell ratio for the treated tissue culture increases (such as increases by a third threshold) compared to the reference tumor:stroma immune cell ratio. In some embodiments, if the treated tumor:stroma immune cell ratio increases (such as increases by a fourth threshold) compared to the reference tumor:stroma immune cell ratio, the responsiveness is predicted to be clinical response. In some embodiments, the anticancer drug regimen comprises an anticancer agent and/or an immunotherapeutic agent. In some embodiments, the anticancer agent includes a chemotherapeutic agent, such as a cytostatic or cytotoxic agent. In some embodiments, the anticancer agent is a targeted therapeutic agent, such as a targeted antibody or targeted small molecule (e.g., protein inhibitor, such as kinase inhibitor). In some embodiments, the immunotherapeutic agent includes an immunomodulatory agent, e.g., an immune checkpoint inhibitor or an immunostimulatory agent. In some embodiments, determining the ratio of NK cells in a region of tumor cells versus a region of normal stroma in a tumor tissue culture comprises determining the ratio of CD56+ cells in a region of tumor cells versus a region of normal stroma in the tumor tissue culture.

In some embodiments, there is provided a method of predicting responsiveness of an individual having a cancer to administration of an anticancer drug regimen, the method comprising a) obtaining a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell; b) inputting the readout into a predictive model; c) using the predictive model to generate an output; and d) using the output to predict responsiveness of the individual to administration of the anticancer drug regimen. In some embodiments, the method provides an improved and more highly refined basis for assessing responsiveness of an individual having a cancer to administration of an anticancer drug regimen as compared to a corresponding method that does not include an assay for tumor infiltration of an immune cell. In some embodiments, the predictive model comprises a first algorithm that uses each of the assessment scores as input and generates the output. In some embodiments, the first algorithm comprises multiplying each of the input assessment scores with a corresponding weightage coefficient to obtain a plurality of weighted assessment scores; and combining the plurality of weighted assessment scores to generate the output. In some embodiments, the predictive model comprises a first algorithm that uses each of the assessment scores for the first set of a plurality of assays as input and generates a preliminary output, and a second algorithm that uses the preliminary output and each of the assessment scores for the second set of one or more assays as input and generates the output. In some embodiments, the first algorithm comprises multiplying each of the input assessment scores with a corresponding weightage coefficient to obtain a plurality of weighted assessment scores; and combining the plurality of weighted assessment scores to generate the preliminary output. In some embodiments, the preliminary output predicts a primary degree of responsiveness of the individual to administration of the anticancer drug regimen, and the second algorithm comprises adjusting the primary predicted degree of responsiveness based on the input assessment scores to generate the output. In some embodiments, the second set of one or more assays consists of the assay for tumor infiltration of an immune cell, and adjusting the primary predicted degree of responsiveness comprises predicting a secondary degree of responsiveness of the individual to administration of the anticancer drug regimen based on the input assessment score, and 1) adjusting the primary predicted degree of responsiveness by decreasing the predicted degree of responsiveness if the secondary predicted degree of responsiveness is lower than the primary predicted degree of responsiveness and the input assessment score is below a first threshold, thereby generating the output; or 2) adjusting the primary predicted degree of responsiveness by increasing the predicted degree of responsiveness if the secondary predicted degree of responsiveness is greater than the primary predicted degree of responsiveness and the input assessment score is above a second threshold, thereby generating the output. In some embodiments, the output predicts complete clinical response, partial clinical response, or no clinical response of the individual to administration of the anticancer drug regimen. In some embodiments, the output predicts response or no response of the individual to administration of the anticancer drug regimen. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell. In some embodiments, the first set of a plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue culture and/or step a) further comprises preparing the tumor tissue culture by culturing tumor tissue from the individual on the tumor microenvironment platform. In some embodiments, the plurality of assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the anticancer drug regimen comprises an anticancer agent and/or an immunotherapeutic agent. In some embodiments, the anticancer agent includes a cytostatic or cytotoxic agent. In some embodiments, the anticancer agent includes a targeted anticancer agent, such as a targeted antibody or targeted small molecule (e.g., protein inhibitor, such as kinase inhibitor). In some embodiments, the anticancer agent includes adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the immunotherapeutic agent includes an immunomodulatory agent, e.g., an immune checkpoint inhibitor or an immunostimulatory agent. In some embodiments, the immunotherapeutic agent includes nivolumab, ipilimumab, pembrolizumab, atezolizumab, and any combination thereof. In some embodiments, the individual is human.

In some embodiments, there is provided a method of predicting responsiveness of an individual having a cancer to administration of an anticancer drug regimen, the method comprising a) obtaining a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, and wherein the plurality of assays comprises an assay for tumor infiltration of an immune cell; b) inputting the readout into a predictive model; c) using the predictive model to generate an output; and d) using the output to predict responsiveness of the individual to administration of the anticancer drug regimen, wherein the predictive model comprises an algorithm that uses each of the assessment scores as input and generates the output. In some embodiments, the algorithm comprises multiplying each of the input assessment scores with a corresponding weightage coefficient to obtain a plurality of weighted assessment scores; and combining the plurality of weighted assessment scores to generate the output. In some embodiments, the output predicts complete clinical response, partial clinical response, or no clinical response of the individual to administration of the anticancer drug regimen. In some embodiments, the output predicts response or no response of the individual to administration of the anticancer drug regimen. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell. In some embodiments, the plurality of assays includes cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue culture and/or step a) further comprises preparing the tumor tissue culture by culturing tumor tissue from the individual on the tumor microenvironment platform. In some embodiments, the plurality of assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the anticancer drug regimen comprises an anticancer agent and/or an immunotherapeutic agent. In some embodiments, the anticancer agent includes a cytostatic or cytotoxic agent. In some embodiments, the anticancer agent includes a targeted anticancer agent, such as a targeted antibody or targeted small molecule (e.g., protein inhibitor, such as kinase inhibitor). In some embodiments, the anticancer agent includes adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the immunotherapeutic agent includes an immunomodulatory agent, e.g., an immune checkpoint inhibitor or an immunostimulatory agent. In some embodiments, the immunotherapeutic agent includes nivolumab, ipilimumab, pembrolizumab, atezolizumab, and any combination thereof. In some embodiments, the individual is human.

In some embodiments, there is provided a method of predicting responsiveness of an individual having a cancer to administration of an anticancer drug regimen, the method comprising a) obtaining a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell; b) inputting the readout into a predictive model; c) using the predictive model to generate an output; and d) using the output to predict responsiveness of the individual to administration of the anticancer drug regimen, wherein the predictive model comprises a first algorithm that uses each of the assessment scores for the first set of a plurality of assays as input and generates a preliminary output, and a second algorithm that uses the preliminary output and each of the assessment scores for the second set of one or more assays as input and generates the output. In some embodiments, the first algorithm comprises multiplying each of the input assessment scores with a corresponding weightage coefficient to obtain a plurality of weighted assessment scores; and combining the plurality of weighted assessment scores to generate the preliminary output. In some embodiments, the preliminary output predicts a primary degree of responsiveness of the individual to administration of the anticancer drug regimen, and the second algorithm comprises adjusting the primary predicted degree of responsiveness based on the input assessment scores to generate the output. In some embodiments, the second set of one or more assays consists of the assay for tumor infiltration of an immune cell, and adjusting the primary predicted degree of responsiveness comprises predicting a secondary degree of responsiveness of the individual to administration of the anticancer drug regimen based on the input assessment score, and 1) adjusting the primary predicted degree of responsiveness by decreasing the predicted degree of responsiveness if the secondary predicted degree of responsiveness is lower than the primary predicted degree of responsiveness and the input assessment score is below a first threshold, thereby generating the output; or 2) adjusting the primary predicted degree of responsiveness by increasing the predicted degree of responsiveness if the secondary predicted degree of responsiveness is greater than the primary predicted degree of responsiveness and the input assessment score is above a second threshold, thereby generating the output. In some embodiments, the output predicts complete clinical response, partial clinical response, or no clinical response of the individual to administration of the anticancer drug regimen. In some embodiments, the output predicts response or no response of the individual to administration of the anticancer drug regimen. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell. In some embodiments, the first set of a plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue culture and/or step a) further comprises preparing the tumor tissue culture by culturing tumor tissue from the individual on the tumor microenvironment platform. In some embodiments, the plurality of assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the anticancer drug regimen comprises an anticancer agent and/or an immunotherapeutic agent. In some embodiments, the anticancer agent includes a cytostatic or cytotoxic agent. In some embodiments, the anticancer agent includes a targeted anticancer agent, such as a targeted antibody or targeted small molecule (e.g., protein inhibitor, such as kinase inhibitor). In some embodiments, the anticancer agent includes adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the immunotherapeutic agent includes an immunomodulatory agent, e.g., an immune checkpoint inhibitor or an immunostimulatory agent. In some embodiments, the immunotherapeutic agent includes nivolumab, ipilimumab, pembrolizumab, atezolizumab, and any combination thereof. In some embodiments, the individual is human.

In some embodiments, there is provided a method of classifying likely responsiveness of an individual having a cancer to administration of an anticancer drug regimen, comprising a) obtaining a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell; b) inputting the readout into a predictive model; c) using the predictive model to generate an output; and d) using the output to classify the likely responsiveness of the individual to administration of the anticancer drug regimen. In some embodiments, the predictive model comprises a first algorithm that uses each of the assessment scores as input and generates the output. In some embodiments, the first algorithm comprises multiplying each of the input assessment scores with a corresponding weightage coefficient to obtain a plurality of weighted assessment scores; and combining the plurality of weighted assessment scores to generate the output. In some embodiments, the predictive model comprises a first algorithm that uses each of the assessment scores for the first set of a plurality of assays as input and generates a preliminary output, and a second algorithm that uses the preliminary output and each of the assessment scores for the second set of one or more assays as input and generates the output. In some embodiments, the first algorithm comprises multiplying each of the input assessment scores with a corresponding weightage coefficient to obtain a plurality of weighted assessment scores; and combining the plurality of weighted assessment scores to generate the preliminary output. In some embodiments, the preliminary output classifies a primary degree of responsiveness of the individual to administration of the anticancer drug regimen, and the second algorithm comprises adjusting the primary classified degree of responsiveness based on the input assessment scores to generate the output. In some embodiments, the second set of one or more assays consists of the assay for tumor infiltration of an immune cell, and adjusting the primary classified degree of responsiveness comprises classifying a secondary degree of responsiveness of the individual to administration of the anticancer drug regimen based on the input assessment score, and 1) adjusting the primary classified degree of responsiveness by decreasing the classified degree of responsiveness if the secondary classified degree of responsiveness is lower than the primary classified degree of responsiveness and the input assessment score is below a first threshold, thereby generating the output; or 2) adjusting the primary classified degree of responsiveness by increasing the classified degree of responsiveness if the secondary classified degree of responsiveness is greater than the primary classified degree of responsiveness and the input assessment score is above a second threshold, thereby generating the output. In some embodiments, the output classifies complete clinical response, partial clinical response, or no clinical response of the individual to administration of the anticancer drug regimen. In some embodiments, the output classifies response or no response of the individual to administration of the anticancer drug regimen. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell. In some embodiments, the first set of a plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue culture and/or step a) further comprises preparing the tumor tissue culture by culturing tumor tissue from the individual on the tumor microenvironment platform. In some embodiments, the plurality of assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the anticancer drug regimen comprises an anticancer agent and/or an immunotherapeutic agent. In some embodiments, the anticancer agent includes a cytostatic or cytotoxic agent. In some embodiments, the anticancer agent includes a targeted anticancer agent, such as a targeted antibody or targeted small molecule (e.g., protein inhibitor, such as kinase inhibitor). In some embodiments, the anticancer agent includes adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the immunotherapeutic agent includes an immunomodulatory agent, e.g., an immune checkpoint inhibitor or an immunostimulatory agent. In some embodiments, the immunotherapeutic agent includes nivolumab, ipilimumab, pembrolizumab, atezolizumab, and any combination thereof. In some embodiments, the individual is human.

In some embodiments, there is provided a computer-implemented method for predicting responsiveness of an individual having a cancer to administration of an anticancer drug regimen, the method comprising a) accessing a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell; b) inputting the readout into a predictive model; c) using the predictive model to generate an output; and d) using the output to predict responsiveness of the individual to administration of the anticancer drug regimen. In some embodiments, the predictive model comprises a first algorithm that uses each of the assessment scores as input and generates the output. In some embodiments, the first algorithm comprises multiplying each of the input assessment scores with a corresponding weightage coefficient to obtain a plurality of weighted assessment scores; and combining the plurality of weighted assessment scores to generate the output. In some embodiments, the predictive model comprises a first algorithm that uses each of the assessment scores for the first set of a plurality of assays as input and generates a preliminary output, and a second algorithm that uses the preliminary output and each of the assessment scores for the second set of one or more assays as input and generates the output. In some embodiments, the first algorithm comprises multiplying each of the input assessment scores with a corresponding weightage coefficient to obtain a plurality of weighted assessment scores; and combining the plurality of weighted assessment scores to generate the preliminary output. In some embodiments, the preliminary output predicts a primary degree of responsiveness of the individual to administration of the anticancer drug regimen, and the second algorithm comprises adjusting the primary predicted degree of responsiveness based on the input assessment scores to generate the output. In some embodiments, the second set of one or more assays consists of the assay for tumor infiltration of an immune cell, and adjusting the primary predicted degree of responsiveness comprises predicting a secondary degree of responsiveness of the individual to administration of the anticancer drug regimen based on the input assessment score, and 1) adjusting the primary predicted degree of responsiveness by decreasing the predicted degree of responsiveness if the secondary predicted degree of responsiveness is lower than the primary predicted degree of responsiveness and the input assessment score is below a first threshold, thereby generating the output; or 2) adjusting the primary predicted degree of responsiveness by increasing the predicted degree of responsiveness if the secondary predicted degree of responsiveness is greater than the primary predicted degree of responsiveness and the input assessment score is above a second threshold, thereby generating the output. In some embodiments, the output predicts complete clinical response, partial clinical response, or no clinical response of the individual to administration of the anticancer drug regimen. In some embodiments, the output predicts response or no response of the individual to administration of the anticancer drug regimen. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell. In some embodiments, the first set of a plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue culture and/or step a) further comprises preparing the tumor tissue culture by culturing tumor tissue from the individual on the tumor microenvironment platform. In some embodiments, the plurality of assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the anticancer drug regimen comprises an anticancer agent and/or an immunotherapeutic agent. In some embodiments, the anticancer agent includes a cytostatic or cytotoxic agent. In some embodiments, the anticancer agent includes a targeted anticancer agent, such as a targeted antibody or targeted small molecule (e.g., protein inhibitor, such as kinase inhibitor). In some embodiments, the anticancer agent includes adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the immunotherapeutic agent includes an immunomodulatory agent, e.g., an immune checkpoint inhibitor or an immunostimulatory agent. In some embodiments, the immunotherapeutic agent includes nivolumab, ipilimumab, pembrolizumab, atezolizumab, and any combination thereof. In some embodiments, the individual is human.

In some embodiments, there is provided a non-transitory computer-readable storage medium storing computer executable instructions that when executed by a computer control the computer to perform a method for predicting responsiveness of an individual having a cancer to administration of an anticancer drug regimen, the method comprising a) accessing a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell; b) inputting the readout into a predictive model; c) receiving, from the predictive model, an output; and d) using the output to predict responsiveness of the individual to administration of the anticancer drug regimen. In some embodiments, the predictive model comprises a first algorithm that uses each of the assessment scores as input and generates the output. In some embodiments, the first algorithm comprises multiplying each of the input assessment scores with a corresponding weightage coefficient to obtain a plurality of weighted assessment scores; and combining the plurality of weighted assessment scores to generate the output. In some embodiments, the predictive model comprises a first algorithm that uses each of the assessment scores for the first set of a plurality of assays as input and generates a preliminary output, and a second algorithm that uses the preliminary output and each of the assessment scores for the second set of one or more assays as input and generates the output. In some embodiments, the first algorithm comprises multiplying each of the input assessment scores with a corresponding weightage coefficient to obtain a plurality of weighted assessment scores; and combining the plurality of weighted assessment scores to generate the preliminary output. In some embodiments, the preliminary output predicts a primary degree of responsiveness of the individual to administration of the anticancer drug regimen, and the second algorithm comprises adjusting the primary predicted degree of responsiveness based on the input assessment scores to generate the output. In some embodiments, the second set of one or more assays consists of the assay for tumor infiltration of an immune cell, and adjusting the primary predicted degree of responsiveness comprises predicting a secondary degree of responsiveness of the individual to administration of the anticancer drug regimen based on the input assessment score, and 1) adjusting the primary predicted degree of responsiveness by decreasing the predicted degree of responsiveness if the secondary predicted degree of responsiveness is lower than the primary predicted degree of responsiveness and the input assessment score is below a first threshold, thereby generating the output; or 2) adjusting the primary predicted degree of responsiveness by increasing the predicted degree of responsiveness if the secondary predicted degree of responsiveness is greater than the primary predicted degree of responsiveness and the input assessment score is above a second threshold, thereby generating the output. In some embodiments, the output predicts complete clinical response, partial clinical response, or no clinical response of the individual to administration of the anticancer drug regimen. In some embodiments, the output predicts response or no response of the individual to administration of the anticancer drug regimen. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell. In some embodiments, the first set of a plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue culture and/or step a) further comprises preparing the tumor tissue culture by culturing tumor tissue from the individual on the tumor microenvironment platform. In some embodiments, the plurality of assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the anticancer drug regimen comprises an anticancer agent and/or an immunotherapeutic agent. In some embodiments, the anticancer agent includes a cytostatic or cytotoxic agent. In some embodiments, the anticancer agent includes a targeted anticancer agent, such as a targeted antibody or targeted small molecule (e.g., protein inhibitor, such as kinase inhibitor). In some embodiments, the anticancer agent includes adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the immunotherapeutic agent includes an immunomodulatory agent, e.g., an immune checkpoint inhibitor or an immunostimulatory agent. In some embodiments, the immunotherapeutic agent includes nivolumab, ipilimumab, pembrolizumab, atezolizumab, and any combination thereof. In some embodiments, the individual is human.

In some embodiments, there is provided a system for generating a report of the predicted responsiveness of an individual having a cancer to administration of an anticancer drug regimen comprising: a) at least one computer database comprising: a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell; and b) a computer-readable program code comprising instructions to: i) input the readout into a predictive model; ii) receive, from the predictive model, an output; iii) use the output to predict responsiveness of the individual to administration of the anticancer drug regimen; and iv) generate a report that comprises the predicted responsiveness of the individual to administration of the anticancer drug regimen. In some embodiments, the predictive model comprises a first algorithm that uses each of the assessment scores as input and generates the output. In some embodiments, the first algorithm comprises multiplying each of the input assessment scores with a corresponding weightage coefficient to obtain a plurality of weighted assessment scores; and combining the plurality of weighted assessment scores to generate the output. In some embodiments, the predictive model comprises a first algorithm that uses each of the assessment scores for the first set of a plurality of assays as input and generates a preliminary output, and a second algorithm that uses the preliminary output and each of the assessment scores for the second set of one or more assays as input and generates the output. In some embodiments, the first algorithm comprises multiplying each of the input assessment scores with a corresponding weightage coefficient to obtain a plurality of weighted assessment scores; and combining the plurality of weighted assessment scores to generate the preliminary output. In some embodiments, the preliminary output predicts a primary degree of responsiveness of the individual to administration of the anticancer drug regimen, and the second algorithm comprises adjusting the primary predicted degree of responsiveness based on the input assessment scores to generate the output. In some embodiments, the second set of one or more assays consists of the assay for tumor infiltration of an immune cell, and adjusting the primary predicted degree of responsiveness comprises predicting a secondary degree of responsiveness of the individual to administration of the anticancer drug regimen based on the input assessment score, and 1) adjusting the primary predicted degree of responsiveness by decreasing the predicted degree of responsiveness if the secondary predicted degree of responsiveness is lower than the primary predicted degree of responsiveness and the input assessment score is below a first threshold, thereby generating the output; or 2) adjusting the primary predicted degree of responsiveness by increasing the predicted degree of responsiveness if the secondary predicted degree of responsiveness is greater than the primary predicted degree of responsiveness and the input assessment score is above a second threshold, thereby generating the output. In some embodiments, the output predicts complete clinical response, partial clinical response, or no clinical response of the individual to administration of the anticancer drug regimen. In some embodiments, the output predicts response or no response of the individual to administration of the anticancer drug regimen. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell. In some embodiments, the first set of a plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue culture and/or step a) further comprises preparing the tumor tissue culture by culturing tumor tissue from the individual on the tumor microenvironment platform. In some embodiments, the plurality of assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the anticancer drug regimen comprises an anticancer agent and/or an immunotherapeutic agent. In some embodiments, the anticancer agent includes a cytostatic or cytotoxic agent. In some embodiments, the anticancer agent includes a targeted anticancer agent, such as a targeted antibody or targeted small molecule (e.g., protein inhibitor, such as kinase inhibitor). In some embodiments, the anticancer agent includes adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the immunotherapeutic agent includes an immunomodulatory agent, e.g., an immune checkpoint inhibitor or an immunostimulatory agent. In some embodiments, the immunotherapeutic agent includes nivolumab, ipilimumab, pembrolizumab, atezolizumab, and any combination thereof. In some embodiments, the individual is human.

In some embodiments, there is provided an assay method comprising a) conducting a plurality of assays on a tumor tissue culture treated with an anticancer drug regimen, wherein the tumor tissue culture comprises a tumor tissue from an individual cultured on a tumor microenvironment platform, wherein the plurality of assays comprises a first set of a plurality of assays and a second set of one or more assays, and wherein the second set of one or more assays comprises an assay for tumor infiltration of an immune cell; and b) generating a readout comprising an assessment score for each of the plurality of assays, wherein the readout is used to predict responsiveness of the individual to administration of the anticancer drug regimen. In some embodiments, using the readout to predict responsiveness of the individual to administration of the anticancer drug regimen comprises c) inputting the readout into a predictive model; d) using the predictive model to generate an output; and e) using the output to predict responsiveness of the individual to administration of the anticancer drug regimen. In some embodiments, the predictive model comprises a first algorithm that uses each of the assessment scores as input and generates the output. In some embodiments, the first algorithm comprises multiplying each of the input assessment scores with a corresponding weightage coefficient to obtain a plurality of weighted assessment scores; and combining the plurality of weighted assessment scores to generate the output. In some embodiments, the predictive model comprises a first algorithm that uses each of the assessment scores for the first set of a plurality of assays as input and generates a preliminary output, and a second algorithm that uses the preliminary output and each of the assessment scores for the second set of one or more assays as input and generates the output. In some embodiments, the first algorithm comprises multiplying each of the input assessment scores with a corresponding weightage coefficient to obtain a plurality of weighted assessment scores; and combining the plurality of weighted assessment scores to generate the preliminary output. In some embodiments, the preliminary output predicts a primary degree of responsiveness of the individual to administration of the anticancer drug regimen, and the second algorithm comprises adjusting the primary predicted degree of responsiveness based on the input assessment scores to generate the output. In some embodiments, the second set of one or more assays consists of the assay for tumor infiltration of an immune cell, and adjusting the primary predicted degree of responsiveness comprises predicting a secondary degree of responsiveness of the individual to administration of the anticancer drug regimen based on the input assessment score, and 1) adjusting the primary predicted degree of responsiveness by decreasing the predicted degree of responsiveness if the secondary predicted degree of responsiveness is lower than the primary predicted degree of responsiveness and the input assessment score is below a first threshold, thereby generating the output; or 2) adjusting the primary predicted degree of responsiveness by increasing the predicted degree of responsiveness if the secondary predicted degree of responsiveness is greater than the primary predicted degree of responsiveness and the input assessment score is above a second threshold, thereby generating the output. In some embodiments, the output predicts complete clinical response, partial clinical response, or no clinical response of the individual to administration of the anticancer drug regimen. In some embodiments, the output predicts response or no response of the individual to administration of the anticancer drug regimen. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell. In some embodiments, the first set of a plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises preparing the tumor tissue culture by culturing tumor tissue from the individual on the tumor microenvironment platform. In some embodiments, the plurality of assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the anticancer drug regimen comprises an anticancer agent and/or an immunotherapeutic agent. In some embodiments, the anticancer agent includes a cytostatic or cytotoxic agent. In some embodiments, the anticancer agent includes a targeted anticancer agent, such as a targeted antibody or targeted small molecule (e.g., protein inhibitor, such as kinase inhibitor). In some embodiments, the anticancer agent includes adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the immunotherapeutic agent includes an immunomodulatory agent, e.g., an immune checkpoint inhibitor or an immunostimulatory agent. In some embodiments, the immunotherapeutic agent includes nivolumab, ipilimumab, pembrolizumab, atezolizumab, and any combination thereof. In some embodiments, the individual is human.

In some embodiments, according to any of the methods described herein, the method provides an improved and more highly refined basis for assessing responsiveness of an individual having a cancer to administration of an anticancer drug regimen as compared to a corresponding method that does not include an assay for tumor infiltration of an immune cell.

### Treatment

In some embodiments, there is provided a method of treating cancer in an individual in need thereof, the method comprising: a) obtaining a readout comprising an assessment score for each of a plurality of assays conducted on a tumor tissue culture treated with an anticancer drug regimen, wherein the tumor tissue culture comprises a tumor tissue from the individual cultured on a tumor microenvironment platform; b) converting the readout into a sensitivity index; c) using the sensitivity index to predict responsiveness to the anticancer drug regimen, wherein at least one of the plurality of assays does not relate to a tumor cell phenotype; and d) administering the anticancer drug regimen to the individual if the individual is predicted to respond to the anticancer drug regimen. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and immune cell contexture assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the anticancer drug regimen comprises an anticancer agent and/or an immunotherapeutic agent. In some embodiments, the anticancer agent includes a chemotherapeutic agent, such as a cytostatic or cytotoxic agent. In some embodiments, the anticancer agent is a targeted therapeutic agent, such as a targeted antibody or targeted small molecule (e.g., protein inhibitor, such as kinase inhibitor). In some embodiments, the immunotherapeutic agent includes an immunomodulatory agent, e.g., an immune checkpoint inhibitor or an immunostimulatory agent.

In some embodiments, there is provided a method treating cancer in an individual in need thereof, the method comprising: a) conducting a plurality of assays on a tumor tissue culture treated with an anticancer drug regimen, wherein the tumor tissue culture comprises a tumor tissue from the individual cultured on a tumor microenvironment platform, and obtaining a readout comprising an assessment score for each of the plurality of assays; b) converting the readout into a sensitivity index; c) using the sensitivity index to predict responsiveness to the anticancer drug regimen, wherein at least one of the plurality of assays does not relate to a tumor cell phenotype; and d) administering the anticancer drug regimen to the individual if the individual is predicted to respond to the anticancer drug regimen. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and immune cell contexture assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the anticancer drug regimen comprises an anticancer agent and/or an immunotherapeutic agent. In some embodiments, the anticancer agent includes a chemotherapeutic agent, such as a cytostatic or cytotoxic agent. In some embodiments, the anticancer agent is a targeted therapeutic agent, such as a targeted antibody or targeted small molecule (e.g., protein inhibitor, such as kinase inhibitor). In some embodiments, the immunotherapeutic agent includes an immunomodulatory agent, e.g., an immune checkpoint inhibitor or an immunostimulatory agent.

In some embodiments, there is provided a method of treating cancer in an individual in need thereof, the method comprising: a) preparing a tumor tissue culture by culturing a tumor tissue from the individual on a tumor microenvironment platform; b) conducting a plurality of assays on the tumor tissue culture that has been treated with an anticancer drug regimen and obtaining a readout comprising an assessment score for each of the plurality of assays; c) converting the readout into a sensitivity index; d) using the sensitivity index to predict responsiveness to the anticancer drug regimen, wherein at least one of the plurality of assays does not relate to a tumor cell phenotype; and e) administering the anticancer drug regimen to the individual if the individual is predicted to respond to the anticancer drug regimen. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and immune cell contexture assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the anticancer drug regimen comprises an anticancer agent and/or an immunotherapeutic agent. In some embodiments, the anticancer agent includes a chemotherapeutic agent, such as a cytostatic or cytotoxic agent. In some embodiments, the anticancer agent is a targeted therapeutic agent, such as a targeted antibody or targeted small molecule (e.g., protein inhibitor, such as kinase inhibitor). In some embodiments, the immunotherapeutic agent includes an immunomodulatory agent, e.g., an immune checkpoint inhibitor or an immunostimulatory agent.

In some embodiments, there is provided a method of treating cancer in an individual in need thereof, the method comprising: a) obtaining a readout comprising an assessment score for each of a plurality of assays conducted on a tumor tissue culture treated with an anticancer drug regimen, wherein the tumor tissue culture comprises a tumor tissue from the individual cultured on a tumor microenvironment platform; b) converting the readout into a sensitivity index; c) determining the ratio of an immune cell (e.g., NK cells) in a region of tumor cells versus a region of normal stroma in the tumor tissue culture, thereby generating a tumor:stroma immune cell ratio; d) using the sensitivity index and the tumor:stroma immune cell ratio to predict responsiveness to the anticancer drug regimen; and e) administering the anticancer drug regimen to the individual if the individual is predicted to respond to the anticancer drug regimen. In some embodiments, the immune cell is an NK cell. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises one or more of serum, plasma, and PBNCs. In some embodiments, at least one of the serum, plasma, and PBNCs are autologous to the individual. In some embodiments, at least one of the serum, plasma, and PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and immune cell contexture assays. In some embodiments, the assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step c) further comprises determining the ratio of the immune cell in a region of tumor cells versus a region of normal stroma in the reference tumor tissue culture, thereby generating a tumor:stroma immune cell ratio for the reference tumor tissue culture. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values. In some embodiments, using the sensitivity index and the tumor:stroma immune cell ratio to predict responsiveness comprises predicting the responsiveness to be the degree of responsiveness associated with the range of values in which the sensitivity index lies if the treated tumor:stroma immune cell ratio does not decrease compared to the reference tumor:stroma immune cell ratio. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, using the sensitivity index and the tumor:stroma immune cell ratio to predict responsiveness comprises predicting the responsiveness to be a lesser degree of responsiveness than that associated with the range of values in which the sensitivity index lies if the treated tumor:stroma immune cell ratio decreases (such as decreases by a first threshold) compared to the reference tumor:stroma immune cell ratio. In some embodiments, if the treated tumor:stroma immune cell ratio decreases (such as decreases by a second threshold) compared to the reference tumor:stroma immune cell ratio, the responsiveness is predicted to be no clinical response. In some embodiments, using the sensitivity index and the tumor:stroma immune cell ratio to predict responsiveness comprises predicting the responsiveness to be a greater degree of responsiveness than that associated with the range of values in which the sensitivity index lies if the treated tumor:stroma immune cell ratio increases (such as increases by a third threshold) compared to the reference tumor:stroma immune cell ratio. In some embodiments, if the treated tumor:stroma immune cell ratio increases (such as increases by a fourth threshold) compared to the reference tumor:stroma immune cell ratio, the responsiveness is predicted to be clinical response. In some embodiments, the anticancer drug regimen comprises an anticancer agent and/or an immunotherapeutic agent. In some embodiments, the anticancer agent includes a chemotherapeutic agent, such as a cytostatic or cytotoxic agent. In some embodiments, the anticancer agent is a targeted therapeutic agent, such as a targeted antibody or targeted small molecule (e.g., protein inhibitor, such as kinase inhibitor). In some embodiments, the immunotherapeutic agent includes an immunomodulatory agent, e.g., an immune checkpoint inhibitor or an immunostimulatory agent. In some embodiments, determining the ratio of NK cells in a region of tumor cells versus a region of normal stroma in a tumor tissue culture comprises determining the ratio of CD56+ cells in a region of tumor cells versus a region of normal stroma in the tumor tissue culture.

In some embodiments, there is provided a method of treating cancer in an individual in need thereof, the method comprising: a) obtaining a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell; b) inputting the readout into a predictive model; c) using the predictive model to generate an output; d) using the output to predict responsiveness of the individual to administration of the anticancer drug regimen; and e) administering the anticancer drug regimen to the individual if the individual is predicted to respond to the anticancer drug regimen. In some embodiments, the method provides an improved and more highly refined basis for assessing responsiveness of an individual having a cancer to administration of an anticancer drug regimen as compared to a corresponding method that does not include an assay for tumor infiltration of an immune cell. In some embodiments, the predictive model comprises a first algorithm that uses each of the assessment scores as input and generates the output. In some embodiments, the first algorithm comprises multiplying each of the input assessment scores with a corresponding weightage coefficient to obtain a plurality of weighted assessment scores; and combining the plurality of weighted assessment scores to generate the output. In some embodiments, the predictive model comprises a first algorithm that uses each of the assessment scores for the first set of a plurality of assays as input and generates a preliminary output, and a second algorithm that uses the preliminary output and each of the assessment scores for the second set of one or more assays as input and generates the output. In some embodiments, the first algorithm comprises multiplying each of the input assessment scores with a corresponding weightage coefficient to obtain a plurality of weighted assessment scores; and combining the plurality of weighted assessment scores to generate the preliminary output. In some embodiments, the preliminary output predicts a primary degree of responsiveness of the individual to administration of the anticancer drug regimen, and the second algorithm comprises adjusting the primary predicted degree of responsiveness based on the input assessment scores to generate the output. In some embodiments, the second set of one or more assays consists of the assay for tumor infiltration of an immune cell, and adjusting the primary predicted degree of responsiveness comprises predicting a secondary degree of responsiveness of the individual to administration of the anticancer drug regimen based on the input assessment score, and 1) adjusting the primary predicted degree of responsiveness by decreasing the predicted degree of responsiveness if the secondary predicted degree of responsiveness is lower than the primary predicted degree of responsiveness and the input assessment score is below a first threshold, thereby generating the output; or 2) adjusting the primary predicted degree of responsiveness by increasing the predicted degree of responsiveness if the secondary predicted degree of responsiveness is greater than the primary predicted degree of responsiveness and the input assessment score is above a second threshold, thereby generating the output. In some embodiments, the output predicts complete clinical response, partial clinical response, or no clinical response of the individual to administration of the anticancer drug regimen. In some embodiments, the output predicts response or no response of the individual to administration of the anticancer drug regimen. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell. In some embodiments, the first set of a plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue culture and/or step a) further comprises preparing the tumor tissue culture by culturing tumor tissue from the individual on the tumor microenvironment platform. In some embodiments, the plurality of assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the anticancer drug regimen comprises an anticancer agent and/or an immunotherapeutic agent. In some embodiments, the anticancer agent includes a cytostatic or cytotoxic agent. In some embodiments, the anticancer agent includes a targeted anticancer agent, such as a targeted antibody or targeted small molecule (e.g., protein inhibitor, such as kinase inhibitor). In some embodiments, the anticancer agent includes adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the immunotherapeutic agent includes an immunomodulatory agent, e.g., an immune checkpoint inhibitor or an immunostimulatory agent. In some embodiments, the immunotherapeutic agent includes nivolumab, ipilimumab, pembrolizumab, atezolizumab, and any combination thereof. In some embodiments, the individual is human.

In some embodiments, there is provided a method of treating cancer in an individual in need thereof, the method comprising: a) obtaining a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, and wherein the plurality of assays comprises an assay for tumor infiltration of an immune cell; b) inputting the readout into a predictive model; c) using the predictive model to generate an output; d) using the output to predict responsiveness of the individual to administration of the anticancer drug regimen; and e) administering the anticancer drug regimen to the individual if the individual is predicted to respond to the anticancer drug regimen, wherein the predictive model comprises an algorithm that uses each of the assessment scores as input and generates the output. In some embodiments, the algorithm comprises multiplying each of the input assessment scores with a corresponding weightage coefficient to obtain a plurality of weighted assessment scores; and combining the plurality of weighted assessment scores to generate the output. In some embodiments, the output predicts complete clinical response, partial clinical response, or no clinical response of the individual to administration of the anticancer drug regimen. In some embodiments, the output predicts response or no response of the individual to administration of the anticancer drug regimen. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell. In some embodiments, the plurality of assays includes cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue culture and/or step a) further comprises preparing the tumor tissue culture by culturing tumor tissue from the individual on the tumor microenvironment platform. In some embodiments, the plurality of assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the anticancer drug regimen comprises an anticancer agent and/or an immunotherapeutic agent. In some embodiments, the anticancer agent includes a cytostatic or cytotoxic agent. In some embodiments, the anticancer agent includes a targeted anticancer agent, such as a targeted antibody or targeted small molecule (e.g., protein inhibitor, such as kinase inhibitor). In some embodiments, the anticancer agent includes adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the immunotherapeutic agent includes an immunomodulatory agent, e.g., an immune checkpoint inhibitor or an immunostimulatory agent. In some embodiments, the immunotherapeutic agent includes nivolumab, ipilimumab, pembrolizumab, atezolizumab, and any combination thereof. In some embodiments, the individual is human.

In some embodiments, there is provided a method of treating cancer in an individual in need thereof, the method comprising: a) obtaining a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell; b) inputting the readout into a predictive model; c) using the predictive model to generate an output; d) using the output to predict responsiveness of the individual to administration of the anticancer drug regimen; and e) administering the anticancer drug regimen to the individual if the individual is predicted to respond to the anticancer drug regimen, wherein the predictive model comprises a first algorithm that uses each of the assessment scores for the first set of a plurality of assays as input and generates a preliminary output, and a second algorithm that uses the preliminary output and each of the assessment scores for the second set of one or more assays as input and generates the output. In some embodiments, the first algorithm comprises multiplying each of the input assessment scores with a corresponding weightage coefficient to obtain a plurality of weighted assessment scores; and combining the plurality of weighted assessment scores to generate the preliminary output. In some embodiments, the preliminary output predicts a primary degree of responsiveness of the individual to administration of the anticancer drug regimen, and the second algorithm comprises adjusting the primary predicted degree of responsiveness based on the input assessment scores to generate the output. In some embodiments, the second set of one or more assays consists of the assay for tumor infiltration of an immune cell, and adjusting the primary predicted degree of responsiveness comprises predicting a secondary degree of responsiveness of the individual to administration of the anticancer drug regimen based on the input assessment score, and 1) adjusting the primary predicted degree of responsiveness by decreasing the predicted degree of responsiveness if the secondary predicted degree of responsiveness is lower than the primary predicted degree of responsiveness and the input assessment score is below a first threshold, thereby generating the output; or 2) adjusting the primary predicted degree of responsiveness by increasing the predicted degree of responsiveness if the secondary predicted degree of responsiveness is greater than the primary predicted degree of responsiveness and the input assessment score is above a second threshold, thereby generating the output. In some embodiments, the output predicts complete clinical response, partial clinical response, or no clinical response of the individual to administration of the anticancer drug regimen. In some embodiments, the output predicts response or no response of the individual to administration of the anticancer drug regimen. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell. In some embodiments, the first set of a plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue culture and/or step a) further comprises preparing the tumor tissue culture by culturing tumor tissue from the individual on the tumor microenvironment platform. In some embodiments, the plurality of assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the anticancer drug regimen comprises an anticancer agent and/or an immunotherapeutic agent. In some embodiments, the anticancer agent includes a cytostatic or cytotoxic agent. In some embodiments, the anticancer agent includes a targeted anticancer agent, such as a targeted antibody or targeted small molecule (e.g., protein inhibitor, such as kinase inhibitor). In some embodiments, the anticancer agent includes adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the immunotherapeutic agent includes an immunomodulatory agent, e.g., an immune checkpoint inhibitor or an immunostimulatory agent. In some embodiments, the immunotherapeutic agent includes nivolumab, ipilimumab, pembrolizumab, atezolizumab, and any combination thereof. In some embodiments, the individual is human.

In some embodiments, there is provided a method of treating cancer in an individual in need thereof, the method comprising: a) obtaining a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell; b) inputting the readout into a predictive model; c) using the predictive model to generate an output; d) using the output to classify the likely responsiveness of the individual to administration of the anticancer drug regimen; and e) administering the anticancer drug regimen to the individual if the individual is classified to respond to the anticancer drug regimen. In some embodiments, the predictive model comprises a first algorithm that uses each of the assessment scores as input and generates the output. In some embodiments, the first algorithm comprises multiplying each of the input assessment scores with a corresponding weightage coefficient to obtain a plurality of weighted assessment scores; and combining the plurality of weighted assessment scores to generate the output. In some embodiments, the predictive model comprises a first algorithm that uses each of the assessment scores for the first set of a plurality of assays as input and generates a preliminary output, and a second algorithm that uses the preliminary output and each of the assessment scores for the second set of one or more assays as input and generates the output. In some embodiments, the first algorithm comprises multiplying each of the input assessment scores with a corresponding weightage coefficient to obtain a plurality of weighted assessment scores; and combining the plurality of weighted assessment scores to generate the preliminary output. In some embodiments, the preliminary output classifies a primary degree of responsiveness of the individual to administration of the anticancer drug regimen, and the second algorithm comprises adjusting the primary classified degree of responsiveness based on the input assessment scores to generate the output. In some embodiments, the second set of one or more assays consists of the assay for tumor infiltration of an immune cell, and adjusting the primary classified degree of responsiveness comprises classifying a secondary degree of responsiveness of the individual to administration of the anticancer drug regimen based on the input assessment score, and 1) adjusting the primary classified degree of responsiveness by decreasing the classified degree of responsiveness if the secondary classified degree of responsiveness is lower than the primary classified degree of responsiveness and the input assessment score is below a first threshold, thereby generating the output; or 2) adjusting the primary classified degree of responsiveness by increasing the classified degree of responsiveness if the secondary classified degree of responsiveness is greater than the primary classified degree of responsiveness and the input assessment score is above a second threshold, thereby generating the output. In some embodiments, the output classifies complete clinical response, partial clinical response, or no clinical response of the individual to administration of the anticancer drug regimen. In some embodiments, the output classifies response or no response of the individual to administration of the anticancer drug regimen. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell. In some embodiments, the first set of a plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue culture and/or step a) further comprises preparing the tumor tissue culture by culturing tumor tissue from the individual on the tumor microenvironment platform. In some embodiments, the plurality of assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the anticancer drug regimen comprises an anticancer agent and/or an immunotherapeutic agent. In some embodiments, the anticancer agent includes a cytostatic or cytotoxic agent. In some embodiments, the anticancer agent includes a targeted anticancer agent, such as a targeted antibody or targeted small molecule (e.g., protein inhibitor, such as kinase inhibitor). In some embodiments, the anticancer agent includes adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the immunotherapeutic agent includes an immunomodulatory agent, e.g., an immune checkpoint inhibitor or an immunostimulatory agent. In some embodiments, the immunotherapeutic agent includes nivolumab, ipilimumab, pembrolizumab, atezolizumab, and any combination thereof. In some embodiments, the individual is human.

In some embodiments, there is provided a method of treating cancer in an individual in need thereof, comprising A) using a computer-implemented method for predicting responsiveness of an individual having a cancer to administration of an anticancer drug regimen, the computer-implemented method comprising a) accessing a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell; b) inputting the readout into a predictive model; c) using the predictive model to generate an output; and d) using the output to predict responsiveness of the individual to administration of the anticancer drug regimen; and B) administering the anticancer drug regimen to the individual if the individual is predicted to respond to the anticancer drug regimen by the computer-implemented method. In some embodiments, the predictive model comprises a first algorithm that uses each of the assessment scores as input and generates the output. In some embodiments, the first algorithm comprises multiplying each of the input assessment scores with a corresponding weightage coefficient to obtain a plurality of weighted assessment scores; and combining the plurality of weighted assessment scores to generate the output. In some embodiments, the predictive model comprises a first algorithm that uses each of the assessment scores for the first set of a plurality of assays as input and generates a preliminary output, and a second algorithm that uses the preliminary output and each of the assessment scores for the second set of one or more assays as input and generates the output. In some embodiments, the first algorithm comprises multiplying each of the input assessment scores with a corresponding weightage coefficient to obtain a plurality of weighted assessment scores; and combining the plurality of weighted assessment scores to generate the preliminary output. In some embodiments, the preliminary output predicts a primary degree of responsiveness of the individual to administration of the anticancer drug regimen, and the second algorithm comprises adjusting the primary predicted degree of responsiveness based on the input assessment scores to generate the output. In some embodiments, the second set of one or more assays consists of the assay for tumor infiltration of an immune cell, and adjusting the primary predicted degree of responsiveness comprises predicting a secondary degree of responsiveness of the individual to administration of the anticancer drug regimen based on the input assessment score, and 1) adjusting the primary predicted degree of responsiveness by decreasing the predicted degree of responsiveness if the secondary predicted degree of responsiveness is lower than the primary predicted degree of responsiveness and the input assessment score is below a first threshold, thereby generating the output; or 2) adjusting the primary predicted degree of responsiveness by increasing the predicted degree of responsiveness if the secondary predicted degree of responsiveness is greater than the primary predicted degree of responsiveness and the input assessment score is above a second threshold, thereby generating the output. In some embodiments, the output predicts complete clinical response, partial clinical response, or no clinical response of the individual to administration of the anticancer drug regimen. In some embodiments, the output predicts response or no response of the individual to administration of the anticancer drug regimen. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell. In some embodiments, the first set of a plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue culture and/or step a) further comprises preparing the tumor tissue culture by culturing tumor tissue from the individual on the tumor microenvironment platform. In some embodiments, the plurality of assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the anticancer drug regimen comprises an anticancer agent and/or an immunotherapeutic agent. In some embodiments, the anticancer agent includes a cytostatic or cytotoxic agent. In some embodiments, the anticancer agent includes a targeted anticancer agent, such as a targeted antibody or targeted small molecule (e.g., protein inhibitor, such as kinase inhibitor). In some embodiments, the anticancer agent includes adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the immunotherapeutic agent includes an immunomodulatory agent, e.g., an immune checkpoint inhibitor or an immunostimulatory agent. In some embodiments, the immunotherapeutic agent includes nivolumab, ipilimumab, pembrolizumab, atezolizumab, and any combination thereof. In some embodiments, the individual is human.

In some embodiments, there is provided a method of treating cancer in an individual in need thereof, comprising A) executing computer executable instructions stored on a non-transitory computer-readable storage medium by a computer to control the computer to perform a method for predicting responsiveness of an individual having a cancer to administration of an anticancer drug regimen, the method for predicting responsiveness comprising a) accessing a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell; b) inputting the readout into a predictive model; c) receiving, from the predictive model, an output; and d) using the output to predict responsiveness of the individual to administration of the anticancer drug regimen; and B) administering the anticancer drug regimen to the individual if the individual is predicted to respond to the anticancer drug regimen by the method of A). In some embodiments, the predictive model comprises a first algorithm that uses each of the assessment scores as input and generates the output. In some embodiments, the first algorithm comprises multiplying each of the input assessment scores with a corresponding weightage coefficient to obtain a plurality of weighted assessment scores; and combining the plurality of weighted assessment scores to generate the output. In some embodiments, the predictive model comprises a first algorithm that uses each of the assessment scores for the first set of a plurality of assays as input and generates a preliminary output, and a second algorithm that uses the preliminary output and each of the assessment scores for the second set of one or more assays as input and generates the output. In some embodiments, the first algorithm comprises multiplying each of the input assessment scores with a corresponding weightage coefficient to obtain a plurality of weighted assessment scores; and combining the plurality of weighted assessment scores to generate the preliminary output. In some embodiments, the preliminary output predicts a primary degree of responsiveness of the individual to administration of the anticancer drug regimen, and the second algorithm comprises adjusting the primary predicted degree of responsiveness based on the input assessment scores to generate the output. In some embodiments, the second set of one or more assays consists of the assay for tumor infiltration of an immune cell, and adjusting the primary predicted degree of responsiveness comprises predicting a secondary degree of responsiveness of the individual to administration of the anticancer drug regimen based on the input assessment score, and 1) adjusting the primary predicted degree of responsiveness by decreasing the predicted degree of responsiveness if the secondary predicted degree of responsiveness is lower than the primary predicted degree of responsiveness and the input assessment score is below a first threshold, thereby generating the output; or 2) adjusting the primary predicted degree of responsiveness by increasing the predicted degree of responsiveness if the secondary predicted degree of responsiveness is greater than the primary predicted degree of responsiveness and the input assessment score is above a second threshold, thereby generating the output. In some embodiments, the output predicts complete clinical response, partial clinical response, or no clinical response of the individual to administration of the anticancer drug regimen. In some embodiments, the output predicts response or no response of the individual to administration of the anticancer drug regimen. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell. In some embodiments, the first set of a plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue culture and/or step a) further comprises preparing the tumor tissue culture by culturing tumor tissue from the individual on the tumor microenvironment platform. In some embodiments, the plurality of assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the anticancer drug regimen comprises an anticancer agent and/or an immunotherapeutic agent. In some embodiments, the anticancer agent includes a cytostatic or cytotoxic agent. In some embodiments, the anticancer agent includes a targeted anticancer agent, such as a targeted antibody or targeted small molecule (e.g., protein inhibitor, such as kinase inhibitor). In some embodiments, the anticancer agent includes adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the immunotherapeutic agent includes an immunomodulatory agent, e.g., an immune checkpoint inhibitor or an immunostimulatory agent. In some embodiments, the immunotherapeutic agent includes nivolumab, ipilimumab, pembrolizumab, atezolizumab, and any combination thereof. In some embodiments, the individual is human.

In some embodiments, there is provided a method of treating cancer in an individual in need thereof, comprising A) using a system to generate a report of the predicted responsiveness of the individual to administration of an anticancer drug regimen, the system comprising: a) at least one computer database comprising: a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell; and b) a computer-readable program code comprising instructions to: i) input the readout into a predictive model; ii) receive, from the predictive model, an output; iii) use the output to predict responsiveness of the individual to administration of the anticancer drug regimen; and iv) generate a report that comprises the predicted responsiveness of the individual to administration of the anticancer drug regimen; and b) administering the anticancer drug regimen to the individual if the individual is predicted to respond to the anticancer drug regimen by the report. In some embodiments, the predictive model comprises a first algorithm that uses each of the assessment scores as input and generates the output. In some embodiments, the first algorithm comprises multiplying each of the input assessment scores with a corresponding weightage coefficient to obtain a plurality of weighted assessment scores; and combining the plurality of weighted assessment scores to generate the output. In some embodiments, the predictive model comprises a first algorithm that uses each of the assessment scores for the first set of a plurality of assays as input and generates a preliminary output, and a second algorithm that uses the preliminary output and each of the assessment scores for the second set of one or more assays as input and generates the output. In some embodiments, the first algorithm comprises multiplying each of the input assessment scores with a corresponding weightage coefficient to obtain a plurality of weighted assessment scores; and combining the plurality of weighted assessment scores to generate the preliminary output. In some embodiments, the preliminary output predicts a primary degree of responsiveness of the individual to administration of the anticancer drug regimen, and the second algorithm comprises adjusting the primary predicted degree of responsiveness based on the input assessment scores to generate the output. In some embodiments, the second set of one or more assays consists of the assay for tumor infiltration of an immune cell, and adjusting the primary predicted degree of responsiveness comprises predicting a secondary degree of responsiveness of the individual to administration of the anticancer drug regimen based on the input assessment score, and 1) adjusting the primary predicted degree of responsiveness by decreasing the predicted degree of responsiveness if the secondary predicted degree of responsiveness is lower than the primary predicted degree of responsiveness and the input assessment score is below a first threshold, thereby generating the output; or 2) adjusting the primary predicted degree of responsiveness by increasing the predicted degree of responsiveness if the secondary predicted degree of responsiveness is greater than the primary predicted degree of responsiveness and the input assessment score is above a second threshold, thereby generating the output. In some embodiments, the output predicts complete clinical response, partial clinical response, or no clinical response of the individual to administration of the anticancer drug regimen. In some embodiments, the output predicts response or no response of the individual to administration of the anticancer drug regimen. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell. In some embodiments, the first set of a plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue culture and/or step a) further comprises preparing the tumor tissue culture by culturing tumor tissue from the individual on the tumor microenvironment platform. In some embodiments, the plurality of assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the anticancer drug regimen comprises an anticancer agent and/or an immunotherapeutic agent. In some embodiments, the anticancer agent includes a cytostatic or cytotoxic agent. In some embodiments, the anticancer agent includes a targeted anticancer agent, such as a targeted antibody or targeted small molecule (e.g., protein inhibitor, such as kinase inhibitor). In some embodiments, the anticancer agent includes adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the immunotherapeutic agent includes an immunomodulatory agent, e.g., an immune checkpoint inhibitor or an immunostimulatory agent. In some embodiments, the immunotherapeutic agent includes nivolumab, ipilimumab, pembrolizumab, atezolizumab, and any combination thereof. In some embodiments, the individual is human.

In some embodiments, there is provided an assay method comprising a) conducting a plurality of assays on a tumor tissue culture treated with an anticancer drug regimen, wherein the tumor tissue culture comprises a tumor tissue from an individual cultured on a tumor microenvironment platform, wherein the plurality of assays comprises a first set of a plurality of assays and a second set of one or more assays, and wherein the second set of one or more assays comprises an assay for tumor infiltration of an immune cell; and b) generating a readout comprising an assessment score for each of the plurality of assays, wherein the readout is used to predict responsiveness of the individual to administration of the anticancer drug regimen, and wherein the anticancer drug regimen is administered to the individual if the individual is predicted to respond to the anticancer drug regimen. In some embodiments, using the readout to predict responsiveness of the individual to administration of the anticancer drug regimen comprises c) inputting the readout into a predictive model; d) using the predictive model to generate an output; and e) using the output to predict responsiveness of the individual to administration of the anticancer drug regimen. In some embodiments, the predictive model comprises a first algorithm that uses each of the assessment scores as input and generates the output. In some embodiments, the first algorithm comprises multiplying each of the input assessment scores with a corresponding weightage coefficient to obtain a plurality of weighted assessment scores; and combining the plurality of weighted assessment scores to generate the output. In some embodiments, the predictive model comprises a first algorithm that uses each of the assessment scores for the first set of a plurality of assays as input and generates a preliminary output, and a second algorithm that uses the preliminary output and each of the assessment scores for the second set of one or more assays as input and generates the output. In some embodiments, the first algorithm comprises multiplying each of the input assessment scores with a corresponding weightage coefficient to obtain a plurality of weighted assessment scores; and combining the plurality of weighted assessment scores to generate the preliminary output. In some embodiments, the preliminary output predicts a primary degree of responsiveness of the individual to administration of the anticancer drug regimen, and the second algorithm comprises adjusting the primary predicted degree of responsiveness based on the input assessment scores to generate the output. In some embodiments, the second set of one or more assays consists of the assay for tumor infiltration of an immune cell, and adjusting the primary predicted degree of responsiveness comprises predicting a secondary degree of responsiveness of the individual to administration of the anticancer drug regimen based on the input assessment score, and 1) adjusting the primary predicted degree of responsiveness by decreasing the predicted degree of responsiveness if the secondary predicted degree of responsiveness is lower than the primary predicted degree of responsiveness and the input assessment score is below a first threshold, thereby generating the output; or 2) adjusting the primary predicted degree of responsiveness by increasing the predicted degree of responsiveness if the secondary predicted degree of responsiveness is greater than the primary predicted degree of responsiveness and the input assessment score is above a second threshold, thereby generating the output. In some embodiments, the output predicts complete clinical response, partial clinical response, or no clinical response of the individual to administration of the anticancer drug regimen. In some embodiments, the output predicts response or no response of the individual to administration of the anticancer drug regimen. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell. In some embodiments, the first set of a plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises preparing the tumor tissue culture by culturing tumor tissue from the individual on the tumor microenvironment platform. In some embodiments, the plurality of assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the anticancer drug regimen. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the anticancer drug regimen comprises an anticancer agent and/or an immunotherapeutic agent. In some embodiments, the anticancer agent includes a cytostatic or cytotoxic agent. In some embodiments, the anticancer agent includes a targeted anticancer agent, such as a targeted antibody or targeted small molecule (e.g., protein inhibitor, such as kinase inhibitor). In some embodiments, the anticancer agent includes adriamycin, gemcitabine, palbociclib, docetaxel, fulvestrant, alpelisib, trametinib, carboplatin, exemestane, everolimus, vinorelbine, olaparib, capecitabine, cyclophosphamide, methotrexate, fluorouracil, mitomycin C, temozolomide, cetuximab, and any combination thereof. In some embodiments, the immunotherapeutic agent includes an immunomodulatory agent, e.g., an immune checkpoint inhibitor or an immunostimulatory agent. In some embodiments, the immunotherapeutic agent includes nivolumab, ipilimumab, pembrolizumab, atezolizumab, and any combination thereof. In some embodiments, the individual is human.

In some embodiments, according to any of the methods described herein, the method provides an improved and more highly refined basis for assessing responsiveness of an individual having a cancer to administration of an anticancer drug regimen as compared to a corresponding method that does not include an assay for tumor infiltration of an immune cell.

### Tumor Microenvironment Platform

to the methods described herein in some embodiments employ a tumor microenvironment platform for culturing tumor tissue, said microenvironment comprising an Extra Cellular Matrix (ECM) composition and culture medium, and optionally including serum, plasma, and/or peripheral blood nuclear cells (PBNCs), such as peripheral blood mononuclear cells (PBMCs). In some embodiments, the tumor microenvironment platform further comprises one or more immune factors. In some embodiments, the tumor microenvironment platform further comprises one or more angiogenic factors. In some embodiments, the tumor microenvironment platform further comprises one or more drugs, such as one or more cancer therapeutic agents (e.g., immunomodulatory agents, such as immune checkpoint inhibitors).

In some embodiments, the serum, plasma, and/or PBNCs are derived from an individual according to any of the methods described herein. For example, according to a method of predicting responsiveness of an individual having a cancer to administration of an anticancer drug regimen described herein, the serum, plasma, and/or PBNCs are derived from the individual (i.e., autologous). In some embodiments, the serum, plasma, and/or PBNCs are not derived from the individual (i.e., heterologous). In some embodiments, the serum and/or plasma is xenogeneic.

In some embodiments, the one or more immune factors are isolated from serum or plasma derived from an individual according to any of the methods described herein (i.e., autologous serum or plasma). In some embodiments, the one or more immune factors are isolated from serum or plasma not derived from the individual (i.e., heterologous serum or plasma). In some embodiments, the serum or plasma is xenogeneic.

In some embodiments, the one or more angiogenic factors are isolated from serum or plasma derived from an individual according to any of the methods described herein (i.e., autologous serum or plasma). In some embodiments, the one or more angiogenic factors are isolated from serum or plasma not derived from the individual (i.e., heterologous serum or plasma). In some embodiments, the serum or plasma is xenogeneic.

In some embodiments, the ECM composition comprises at least three components selected from group consisting of collagen 1, collagen 3, collagen 4, collagen 6, Fibronectin, Vitronectin, Cadherin, Filamin A, Vimentin, Laminin, Decorin, Tenascin C, Osteopontin, Basement membrane proteins, Cytoskeletal proteins and Matrix proteins.

In some embodiments, the components of the ECM composition are specific to tissue from a tumor, and are selected by subjecting a sample of the tumor tissue to one or more assays to identify components of the ECM present in the tumor tissue (e.g., mass spectrometry, such as liquid chromatography-mass spectrometry (LCMS)), and selecting from among the identified ECM components at least three components selected from the group consisting of collagen 1, collagen 3, collagen 4, collagen 6, Fibronectin, Vitronectin, Cadherin, Filamin A, Vimentin, Laminin, Decorin, Tenascin C, Osteopontin, Basement membrane proteins, Cytoskeletal proteins and Matrix proteins. In some embodiments, the tumor is, for example, a stomach, colon, head & neck, brain, oral cavity, breast, gastric, gastro-intestinal, oesophageal, colorectal, pancreatic, lung (e.g., non-small cell lung or small cell lung), liver, kidney, ovarian, uterine, bone, prostate, testicular, thyroid, or bladder tumor. In some embodiments, the tumor is a glioblastoma, astrocytoma, or melanoma. Also contemplated are ECM compositions specific for hematological cancers including AML (Acute Myeloid Leukemia), CML (Chronic Myelogenous Leukemia), ALL (Acute Lymphocytic Leukemia), TALL (T-cell Acute Lymphoblastic Leukemia), NHL (Non-Hodgkins Lymphoma), DBCL (Diffuse B-cell Lymphoma), CLL (Chronic Lymphocytic Leukemia) and multiple myeloma. In some embodiments, the ECM composition comprises ECM components identified from a sample of bone marrow. In some embodiments, the ECM composition comprises ECM components identified from a sample of blood plasma. In some embodiments, the ECM composition comprises ECM components identified from an autologous sample (e.g., the tumor tissue in the tumor microenvironment platform is derived from the same individual as the sample from which the ECM components are identified). In some embodiments, the ECM composition comprises ECM components identified from a heterologous sample (e.g., the tumor tissue in the tumor microenvironment platform is derived from a different individual than the sample from which the ECM components are identified).

In some embodiments, the ECM composition comprises collagen 1 at a concentration ranging from about 0.01 μg/ml to about 100 μg/ml, such as at about 5 μg/ml or about 20 μg/ml or about 50 μg/ml. In some embodiments, the ECM composition comprises collagen 3 at a concentration ranging from about 0.01 μg/ml to about 100 μg/ml, such as at about 0.1 μg/ml or about 1 μg/ml or about 100 μg/ml. In some embodiments, the ECM composition comprises collagen 4 at a concentration ranging from about 0.01 μg/ml to about 500 μg/ml, such as at about 5 μg/ml or about 20 μg/ml or about 250 μg/ml. In some embodiments, the ECM composition comprises collagen 6 at a concentration ranging from about 0.01 μg/ml to about 500 μg/ml, such as at about 0.1 μg/ml or about 1 μg/ml or about 10 μg/ml. In some embodiments, the ECM composition comprises Fibronectin at a concentration ranging from about 0.01 μg/ml to about 750 μg/ml, such as at about 5 μg/ml or about 20 μg/ml or about 500 μg/ml. In some embodiments, the ECM composition comprises Vitronectin at a concentration ranging from about 0.01 μg/ml to about 95 μg/ml, such as at about 5 μg/ml or about 10 μg/ml. In some embodiments, the ECM composition comprises Cadherin at a concentration ranging from about 0.01 μg/ml to about 500 μg/ml, such as at about 1 μg/ml and about 5 μg/ml. In some embodiments, the ECM composition comprises Filamin A at a concentration ranging from about 0.01 μg/ml to about 500 μg/ml, such as at about 5 μg/ml or about 10 μg/ml. In some embodiments, the ECM composition comprises Vimentin at a concentration ranging from about 0.01 μg/ml to about 100 μg/ml, such as at about 1 μg/ml or about 10 μg/ml. In some embodiments, the ECM composition comprises Laminin at a concentration ranging from about 0.01 μg/ml to about 100 μg/ml, such as at about 5 μg/ml or about 10 μg/ml or about 20 μg/ml. In some embodiments, the ECM composition comprises Decorin at concentration ranging from about 0.01 μg/ml to about 100 μg/ml, such as at about 10 μg/ml or about 20 μg/ml. In some embodiments, the ECM composition comprises Tenascin C at a concentration ranging from about 0.01 μg/ml to about 500 μg/ml, such as at about 10 μg/ml or about 25 μg/ml. In some embodiments, the ECM composition comprises Osteopontin at a concentration ranging from about 0.01 μg/ml to about 150 μg/ml, such as at about 1 μg/ml or about 5 μg/ml. In some embodiments, the ECM composition comprises one or more Basement membrane proteins at a concentration ranging from about 0.01 μg/ml to about 150 μg/ml. In some embodiments, the ECM composition comprises one or more cytoskeletal proteins at a concentration ranging from about 0.01 μg/ml to about 150 μg/ml. In some embodiments, the ECM composition comprises one or more matrix proteins at a concentration ranging from about 0.01 μg/ml to about 150 μg/ml.

In some embodiments, the tumor microenvironment platform comprises a substrate coated with the ECM composition. In some embodiments, the substrate is, for example, a plate, base, flask, dish, petriplate, or petridish. The substrate may be made of any material suitable for being coated with the ECM composition. In some embodiments, the substrate is coated with the EMC composition by depositing a liquid mixture comprising the ECM composition on the substrate and allowing the liquid mixture to dry. In some embodiments, the liquid mixture is an aqueous mixture. In some embodiments, the liquid mixture is allowed to dry at a temperature at least about 25 (such as at least about any of 25, 26, 27, 28, 29, 30, 31, 32, 33, 34, 35, 36, 37, 38, 39, 40, 41, 42, or more, including any ranges between these values) ° C. In some embodiments, the substrate is washed with an appropriate solution (e.g., a buffer, such as PBS) at least 1× (such as at least 1×, 2×, 3×, or more) following coating with the ECM composition. In some embodiments, the substrate has been stored at a temperature no greater than about 4 (such as no greater than about any of 4, 0, −5, −10, −15, −20, −25, −30, or less, including any ranges between these values) ° C. prior to combination with culture medium.

In some embodiments, the culture medium is combined with the ECM composition by overlaying the culture medium on a substrate coated with the ECM composition. In some embodiments, the culture medium comprises Dulbecco's Modified Eagle Medium (DMEM) or RPMI1640 (Roswell Park Memorial Institute Medium), for example DMEM or RPMI1640 at a concentration ranging from about 60% to about 100%, such as about 80%. In some embodiments, the culture medium comprises serum, such as heat inactivated FBS (Foetal Bovine Serum), for example FBS at a concentration ranging from about 0.1% to about 40%, such as about 2% wt/wt. In some embodiments, the serum is added to the culture medium after culturing the tumor tissue in the culture medium for a duration of time. In some embodiments, the serum is added to the culture medium after culturing the tumor tissue in the culture medium for at least 6 hours (such as at least about any of 6, 7, 8, 9, 10, 11, 12, 14, 16, 18, 20, 22, or 24 hours or more). In some embodiments, the culture medium comprises Penicillin-Streptomycin at a concentration ranging from about 1% to about 2%, such as about 1% wt/wt. In some embodiments, the culture medium comprises sodium pyruvate at a concentration ranging from about 10 mM to about 500 mM, such as about 100 mM. In some embodiments, the culture medium comprises a nonessential amino acid, including, but not limited to, L-glutamine, at a concentration ranging from about 1 mM to about 10 mM, such as about 5 mM. In some embodiments, the culture medium comprises HEPES ((4-(2-hydroxyethyl)-1-piperazineethanesulfonic acid) at concentration ranging from about 1 mM to about 20 mM, preferably about 10 mM; the serum, is at concentration ranging from about 0.1% to about 10%, preferably about 2%. In some embodiments, the culture medium is exchanged at regular intervals. In some embodiments, the culture medium is exchanged at an interval of at least about 12 hours (such as at least about any of 12, 14, 16, 18, 20, 22, 24, 30, 36, 40, 44, 48, 60, or 72 hours or more).

In some embodiments, the one or more drugs are present in the culture medium before it is combined with the ECM composition. In some embodiments, at least one of the one or more drugs is added to the culture medium after it is combined with the ECM composition. In some embodiments, each of the one or more drugs is added to the culture medium after it is combined with the ECM composition. In some embodiments, at least some of the one or more drugs are added to the culture medium at different times. For example, in some embodiments, at least one of the one or more drugs is added to the culture medium before it is combined with the ECM compositions, and at least one of the one or more drugs is added to the culture medium after it is combined with the ECM composition. In some embodiments, at least some of the one or more drugs are added to the culture medium at different times after it is combined with the ECM composition. In some embodiments, at least some of the one or more drugs are cancer therapeutic agents. In some embodiments, each of the one or more drugs are cancer therapeutic agents. In some embodiments, the one or more drugs comprise a chemotherapeutic agent, such as a cytostatic or cytotoxic agent. In some embodiments, the one or more drugs comprise a targeted cancer therapeutic agent, such as a targeted antibody or targeted small molecule drug (e.g., protein inhibitor, such as kinase inhibitor). In some embodiments, the one or more drugs comprise an immunomodulatory agent, such as an immune checkpoint inhibitor or immunostimulatory agent. In some embodiments, the one or more drugs comprise one or more agents selected from alkylating agents, anthracycline agents, antibodies, cytoskeletal disrupting agents (e.g., taxanes), epothilones, histone deacetylase inhibitors (HDACi), kinase inhibitors, macrolides, nucleotide analogs and precursor analogs, peptide antibiotics, platinum-based agents, retinoids, topoisomerase inhibitors (e.g., topoisomerase I or topoisomerase II inhibitors), and vinca alkaloids and derivatives.

The term “immunomodulatory agent” refers to a therapeutic agent that when present, alters, suppresses or stimulates the body's immune system. Immunomodulators can include compositions or formulations that activate the immune system (e.g., adjuvants or activators), or downregulate the immune system. Adjuvants can include aluminum-based compositions, as well as compositions that include bacterial or mycobacterial cell wall components. Activators can include molecules that activate antigen presenting cells to stimulate the cellular immune response. For example, activators can be immunostimulant peptides. Activators can include, but are not limited to, agonists of toll-like receptors TLR-2, 3, 4, 6, 7, 8, or 9, granulocyte macrophage colony stimulating factor (GM-CSF); TNF; CD40L; CD28; FLT-3 ligand; or cytokines such as IL-1, IL-2, IL-4, IL-7, IL-12, IL-15, or IL-21. Activators can include agonists of activating receptors (including co-stimulatory receptors) on T cells, such as an agonist (e.g., agonistic antibody) of CD28, OX40, ICOS, GITR, 4-1BB, CD27, CD40, or HVEM. Activators can also include compounds that inhibit the activity of an immune suppressor, such as an inhibitor of the immune suppressors IL-10, IL-35, FasL, TGF-β, indoleamine-2,3 dioxygenase (IDO), or cyclophosphamide, or inhibit the activity of an immune checkpoint such as an antagonist (e.g., antagonistic antibody) of CTLA4, PD-1, PD-L1, PD-L2, LAG3, B7-1, B7-H3, B7-H4, BTLA, VISTA, KIR, A2aR, or TIM3. Activators can also include costimulatory molecules such as CD40, CD80, or CD86. Immunomodulators can also include agents that downregulate the immune system such as antibodies against IL-12p70, antagonists of toll-like receptors TLR-2, 3, 4, 5, 6, 8, or 9, or general suppressors of immune function such as cyclophosphamide, cyclosporin A or FK506. Other antibodies of interest include those directed to tumor cell targets, including for example anti-CD38 antibody (such as daratumumab). These agents (e.g., adjuvants, activators, or downregulators) can be combined to shape an optimal immune response.

The term “immune checkpoint inhibitor” refers to compounds that inhibit the activity of control mechanisms of the immune system. Immune system checkpoints, or immune checkpoints, are inhibitory pathways in the immune system that generally act to maintain self-tolerance or modulate the duration and amplitude of physiological immune responses to minimize collateral tissue damage. Immune checkpoint inhibitors can inhibit an immune system checkpoint by inhibiting the activity of a protein in the pathway. Immune system checkpoint proteins include, but are not limited to, cytotoxic T-lymphocyte antigen 4 (CTLA4), programmed cell death 1 protein (PD-1), programmed cell death 1 ligand 1 (PD-L1), programmed cell death 1 ligand 2 (PD-L2), lymphocyte activation gene 3 (LAG3), B7-1, B7-H3, B7-H4, T cell membrane protein 3 (TIM3), B- and T-lymphocyte attenuator (BTLA), V-domain immunoglobulin (Ig)-containing suppressor of T-cell activation (VISTA), Killer-cell immunoglobulin-like receptor (KIR), and A2A adenosine receptor (A2aR). As such, immune checkpoint inhibitors include antagonists of CTLA4, PD-1, PD-L1, PD-L2, LAG3, B7-1, B7-H3, B7-H4, BTLA, VISTA, KIR, A2aR, or TIM3. For example, antibodies that bind to CTLA4, PD-1, PD-L1, PD-L2, LAG3, B7-1, B7-H3, B7-H4, BTLA, VISTA, KIR, A2aR, or TIM3 and antagonize their function are checkpoint inhibitors. Moreover, any molecule (e.g., peptide, nucleic acid, small molecule, etc.) that inhibits the inhibitory function of an immune system checkpoint is an immune checkpoint inhibitor.

In some embodiments, according to any of the methods described herein, the immunomodulatory agent enhances an immune response in the individual and may include, but is not limited to, a cytokine, a chemokine, a stem cell growth factor, a lymphotoxin, an hematopoietic factor, a colony stimulating factor (CSF), erythropoietin, thrombopoietin, tumor necrosis factor-alpha (TNF), TNF-beta, granulocyte-colony stimulating factor (G-CSF), granulocyte macrophage-colony stimulating factor (GM-CSF), interferon-alpha, interferon-beta, interferon-gamma, interferon-lambda, stem cell growth factor designated “Si factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-beta, platelet-growth factor, TGF-alpha, TGF-beta, insulin-like growth factor-I, insulin-like growth factor-II, macrophage-CSF (M-CSF), IL-1, IL-1a, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, lymphotoxin, thalidomide, lenalidomide, or pomalidomide. In some embodiments, the immunomodulator is pomalidomide or an enantiomer or a mixture of enantiomers thereof, or a pharmaceutically acceptable salt, solvate, hydrate, co-crystal, clathrate, or polymorph thereof. In some embodiments, the immunomodulator is lenalidomide or an enantiomer or a mixture of enantiomers thereof, or a pharmaceutically acceptable salt, solvate, hydrate, co-crystal, clathrate, or polymorph thereof.

In some embodiments, according to any of the methods described herein, the immunomodulatory agent enhances an immune response in the individual and may include, but is not limited to, an antagonistic antibody selected from the group consisting of anti-CTLA4 (such as Ipilimumab and Tremelimumab), anti-PD-1 (such as Nivolumab, Pidilizumab, and Pembrolizumab), anti-PD-L1 (such as MPDL3280A, BMS-936559, MEDI4736, and Avelumab), anti-PD-L2, anti-LAG3 (such as BMS-986016 or C9B7W), anti-B7-1, anti-B7-H3 (such as MGA271), anti-B7-H4, anti-TIM3, anti-BTLA, anti-VISTA, anti-KIR (such as Lirilumab and IPH2101), anti-A2aR, anti-CD52 (such as alemtuzumab), anti-IL-10, anti-IL-35, anti-FasL, and anti-TGF-β (such as Fresolumimab). In some embodiments, the antibody is a monoclonal antibody. In some embodiments, the antibody is human or humanized.

In some embodiments, according to any of the methods described herein, the immunomodulator enhances an immune response in the individual and may include, but is not limited to, an agonistic antibody selected from the group consisting of anti-CD28, anti-OX40 (such as MEDI6469), anti-ICOS (such as JTX-2011, Jounce Therapeutics), anti-GITR (such as TRX518), anti-4-1BB (such as BMS-663513 and PF-05082566), anti-CD27 (such as Varlilumab and hCD27.15), anti-CD40 (such as CP870,893), and anti-HVEM. In some embodiments, the antibody is a monoclonal antibody. In some embodiments, the antibody is human or humanized

In some embodiments, the tumor tissue cultured in the tumor microenvironment platform is primary tumor tissue derived from an individual (e.g., a human), such as by standard protocols (e.g., by excision during surgery or by biopsy). In some embodiments, the tumor tissue cultured in the tumor microenvironment platform is from a tumor xenograft derived from primary tumor tissue from a first individual (e.g., a human) that has been implanted (e.g., subcutaneously) in a second individual (e.g., an immune-compromised mouse, such as a SCID mouse). In some embodiments, tumor tissue from a tumor xenograft is excised from the xenograft after it has reached a threshold volume. In some embodiments, the threshold volume is at least about 500 (such as at least about any of 500, 600, 700, 800, 900, 1000, 1200, 1400, 1600, 1800, 2000, or more, including any ranges between these values) mm³. Tumor tissue can be excised according to any of the methods of tumor excision known in the art. In some embodiments, the tumor tissue is a tissue section having a thickness from about 100 μm to about 3000 μm (such as about any of 100, 200, 300, 400, 500, 600, 700, 800, 900, 1000, 1200, 1400, 1600, 1800, 2000, 2200, 2400, 2600, 2800, or 3000 μm, including any ranges between these values).

In some embodiments, there is provided a method of producing a tumor microenvironment platform for culturing tumor tissue, the method comprising coating a substrate with an ECM composition according to any of the embodiments described herein and overlaying the coated substrate with culture medium, optionally along with serum, plasma and/or PBNC (such as autologous serum, plasma and/or PBNCs). In some embodiments, one or more drugs, such as cancer therapeutic agents (e.g., immunomodulatory agents, such as immune checkpoint inhibitors), are included in the culture medium. In some embodiments, the one or more drugs are included in the culture medium prior to overlaying the coated substrate. In some embodiments, the one or more drugs are added to the culture medium after overlaying the coated substrate.

In some embodiments, there is provided a method of organotypic culturing of a tumor tissue, the method comprising culturing the tumor tissue on a tumor microenvironment platform according to any of the embodiments described herein, thereby producing an organotypic culture.

In some embodiments, according to any of the methods described herein, the tumor tissue is obtained from a source selected from the group consisting of central nervous system, bone marrow, blood, spleen, thymus, heart, mammary gland, liver, pancreas, thyroid, skeletal muscle, kidney, lung, intestine, stomach, esophagus, ovary, bladder, testis, uterus, stromal tissue and connective tissue, or any combinations thereof. In some embodiments, the tumor tissue is obtained by excision during surgery or by biopsy (such as punch biopsy). In some embodiments, the tumor tissue is derived from a xenograft implant. In some embodiments, a section of the tumor tissue having a thickness of about 100 μm to about 3000 is used for culturing in the tumor microenvironment platform. In some embodiments, tumor tissue having a volume of about 0.2 cm³ to about 0.5 cm³ is used for culturing in the tumor microenvironment platform.

In some embodiments, according to any of the methods described herein, culturing of the tumor tissue is carried out at a temperature ranging from about 30° C. to about 40° C., such as at about 37° C. In some embodiments, culturing of the tumor tissue is carried out for a duration of time ranging from about 2 days to 10 days, such as about 3 days to 7 days. In some embodiments, culturing of the tumor tissue is carried out at about 5% CO₂.

### Readout Assays

In some embodiments, the plurality of assays used for producing the readout according to any of the methods described herein include both kinetic and end-point assays. In some embodiments, the plurality of assays include cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, immune cell contexture assays, and any combination thereof. In some embodiments, the plurality of assays comprise (such as consist of) no more than 10 assays (such as no more than any of 9, 8, 7, 6, 5, 4, or 3 assays). In some embodiments, the plurality of assays include at least one assay (such as at least any of 2, 3, 4, 5, 6 or more assays) that does not relate to a tumor cell phenotype.

In some embodiments, the assays for cell viability include, for example, MTT assay, WST assay, ATP uptake assay and glucose uptake assay. In some embodiments, the assays for cell proliferation and metabolism include, for example, Ki67 assay, PCNA (proliferating nuclear cell antigen) assay, ATP/ADP ratio assay, and glucose uptake assay. In some embodiments, the assays for cell death include, for example, lactose dehydrogenase (LDH) assay, Activated Caspase 3 assay, Activated Caspase 8 assay, Nitric Oxide Synthase assay, and TUNEL assay. In some embodiments, the assays for senescence include, for example, senescence-associated beta-galactosidase staining. In some embodiments, the assays for tumor morphology and tumor stroma include, for example, Haemaotxylin & Eosin staining (H&E) for tumor cell content, size of the tumor cells, ratio of viable cells/dead cells, ratio of tumor cells/normal cells, tumor/macrophage ratio, nuclear size, density, and integrity, apoptotic bodies, and mitotic figures. In some embodiments, one or more of the plurality of assays is an immunohistochemical assay, including multi-plexed immunohistochemical assays, such as for evaluating simultaneous activity/infiltration of immune cells and/or signaling/activity components. In some embodiments, one or more of the plurality of assays is a quantitative or qualitative assay including, for example, ELISA, blotting (e.g., Western, Northern, or Southern blot), LC/MS, bead based assay, immune-depletion assay, and chromatographic assay. In some embodiments, one or more of the plurality of assays comprises a fluorogenic probe, such as a probe that generates a fluorescent signature following cleavage (e.g., enzymatic cleavage, such as by granzyme, caspase-1, TNFa-converting enzyme (TACE), or matrix metalloprotease) of a substrate.

In some embodiments, the cytokine profile assays include assays for one or more of TGF-β, IFN-γ, IL-6, GM-CSF, IL1b, IL-4, TNFa, IL-23/12, CD40/CD40L, and IL-8. In some embodiments, the cytokine profile assays include one or more immunohistochemical and/or flow cytometric assays for cells expressing the cytokines. In some embodiments, the cytokine profile assays include one or more cytokine secretion assays, such as ELISA-based assays for determining secretion of the cytokines.

In some embodiments, the enzyme activity assays include assays (such as ELISA-based assays) to determine the concentration of enzymes (such as secreted enzymes, e.g., granzyme) in the tumor tissue culture.

In some embodiments, the plurality of assays comprise assays (such as ELISA-based assays) to determine the concentration of cytolytic proteins (such as cytotoxic T cell cytolytic proteins, e.g., perforin) in the tumor tissue culture.

In some embodiments, the immune contexture assays include assays for the presence of specific immune cells, such as T cells (e.g., CD4+ T cells, CD8+ T cells, regulatory T cells, NK T cells) and NK cells, in the tumor microbed (e.g., tumor infiltrating lymphocutes). In some embodiments, the immune contexture assays include assays for the ratio of a specific immune cell (e.g., NK cells or T cells) between an area of tumor cells and an area of normal stroma in the tumor tissue culture. In some embodiments, where the immune cell is an NK cell, the assay comprises determining the ratio of CD56+ cells between an area of tumor cells and an area of normal stroma in the tumor tissue culture. In some embodiments, the immune contexture assays include assays for surface expression of immune checkpoint molecules. In some embodiments, the immune contexture assays include immune response related surface expression assays, such as assays for expression of markers selected from VEGFR, CXCR4, MMP-9, FOXP3, PD-1, PD-L1, CD68, CD3, CD4, CD8, CD34, CD25, CD45, CD127, CTLA4, CEACAM, LAG3, TIM3, ILDR2, OX40, 4-1-BB, and GITR, including immunohistochemical and flow cytometric assays. In some embodiments, the immune contexture assays include assays for the activity of immune cells in the culture, such as granzyme B and perforin release assays (including quantitation assays, such as ELISA-based assays, and activity assays, such as fluorometric assays).

In some embodiments, the immune contexture assays include an assay for tumor infiltration of an immune cell. In some embodiments, the assay for tumor infiltration of an immune cell comprises determining the amount of the immune cell in a region of tumor cells in the tumor tissue culture. In some embodiments, the assay for tumor infiltration of an immune cell comprises determining the ratio of i) the amount of the immune cell in a region of tumor cells in the tumor tissue culture to ii) the amount of the immune cell in a region of normal stroma in the tumor tissue culture. In some embodiments, the immune cell is an NK cell.

In some embodiments, there is provided an assay for tumor infiltration of an immune cell in a tumor tissue culture derived from an individual, comprising determining the ratio of i) the amount of the immune cell in a region of tumor cells in the tumor tissue culture to ii) the amount of the immune cell in a region of normal stroma in the tumor tissue culture. In some embodiments, the tumor tissue culture is treated with an anticancer drug regimen. In some embodiments, the tumor tissue culture is a reference tumor tissue culture. In some embodiments, the reference tumor tissue culture is not treated with the anticancer drug regimen. In some embodiments, the method further comprises determining the change (e.g., fold-change) in the ratio going from the reference tumor tissue to the tumor tissue culture treated with the anticancer drug regimen. In some embodiments, the tumor tissue culture is a tumor tissue culture according to any of the methods described herein. In some embodiments, the tumor tissue culture is prepared according to any of the methods of preparing a tumor tissue culture described herein. In some embodiments, the immune cell is an NK cell.

In some embodiments, there is provided an assay for the change in tumor infiltration of an immune cell in tumor tissue culture derived from an individual under pressure from administration of an anticancer drug regimen, comprising a) determining the ratio of i) the amount of the immune cell in a region of tumor cells in a first tumor tissue culture to ii) the amount of the immune cell in a region of normal stroma in the first tumor tissue culture, wherein the first tumor tissue culture is derived from the individual and is treated with the anticancer drug regimen; b) determining the ratio of i) the amount of the immune cell in a region of tumor cells in a second tumor tissue culture to ii) the amount of the immune cell in a region of normal stroma in the second tumor tissue culture, wherein the second tumor tissue culture is derived from the individual and is not treated with the anticancer drug regimen; and c) determining the change (e.g., fold-change) in the ratio of a) as compared to the ratio of b). In some embodiments, the first and second tumor tissue cultures are, individually, a tumor tissue culture according to any of the tumor tissue cultures described herein. In some embodiments, the first and second tumor tissue cultures are, individually, prepared according to any of the methods of preparing a tumor tissue culture described herein. In some embodiments, the immune cell is an NK cell.

In some embodiments, each of the plurality of assays is assigned a numeric assessment score based on the results of the assay under treated and control conditions. The numeric assessment score can be based on any number of transformations of the assay results into a numeric representation, such as those used conventionally in the art for the particular assay. In some embodiments, the assessment score is determined as the fold change in a numeric output of the assay with treatment as compared to control. For example, in some embodiments, the assay is for determining the amount of a particular cell type (e.g., CD8+ T cell) in the tissue culture as a percent of total cells, with an output of 40% for the treated condition vs 20% for the control condition, and the assessment score is determined as 2, based on the two-fold increase. In some embodiments, the assessment score is determined based on the increase of a numeric output of the assay with treatment as compared to control. For example, in some embodiments, the assay is for determining the amount of a particular cell type (e.g., CD8+ T cell) in the tissue culture as a percent of total cells, with an output of 40% for the treated condition vs 20% for the control condition, and the assessment score is determined as 20, based on the 20% increase. In some embodiments, the assessment score is determined based on the percent inhibition of a numeric output of the assay with treatment as compared to control. For example, in some embodiments, the assay is a viability assay with 70% viability for treatment compared to control, and the assessment score is determined as 30, based on the 30% inhibition in viability. In some embodiments, the assessments scores are determined such that increasing values correspond to increasing degrees of response to treatment. For example, in some embodiments, the assay is a tumor cell viability assay with an assessment score based on an output of % inhibition in tumor cell viability for treatment compared to control, where 100% inhibition is more likely to predict a stronger response to treatment than 0% inhibition. In some embodiments, all of the assessment scores are determined such that they fall within the same predetermined range. For example, in some embodiments, all of the assessment score are determined such that they range between 0 and 100.

### Predictive Model

The methods described herein in some embodiments employ a predictive model used to generate an output for an individual based on assessment scores from assays conducted on tumor tissue explants derived from the individual cultured in a tumor microenvironment platform as described herein, and treated with a drug or combination of drugs. In some embodiments, the output predicts responsiveness of the individual to treatment with the drug or combination of drugs. In some embodiments, the output is used to classify the likely responsiveness of an individual to treatment with the drug or combination of drugs. In some embodiments, the output is a sensitivity index. The terms “sensitivity index” and “M-score” are used herein interchangeably. In some embodiments, the predictive model comprises weightage coefficients for each of the plurality of assays, and the output (e.g., sensitivity index) is generated by multiplying the numeric assessment score of each of the plurality of assays with its weightage score to obtain a weighted assessment score for each of the plurality of assays, and adding together each of the weighted assessment scores to obtain the output (e.g., sensitivity index).

In some embodiments, the weightage coefficients associated with each of the assays used for generating the output (e.g., sensitivity index) in the predictive model are determined using a machine learning algorithm. See Majumder, B., et al. Nature communications. 6, 2015, incorporated by reference herein in its entirety. In some embodiments, tumor tissue samples derived from a number of individuals prior to their treatment with a drug or combination of drugs are used to obtain results from a plurality of tumor tissue explant assays as described herein, which are transformed into numeric assessment scores, and the assessment scores for each individual paired with their associated clinical outcome (e.g., PERCIST/RECIST tumor response metrics, such as complete clinical response, partial clinical response, and no clinical response) following treatment are input into the machine learning algorithm, whereby the machine learning algorithm outputs weightage coefficients for each of the assays such that the sensitivity indices for the number of individual (calculated for each individual by multiplying their assessment score for each of the assays with its associated weightage score to generate weighted assessment scores, and adding together these weighted assessment scores) correlate (e.g., linearly correlate) with their clinical outcome. In some embodiments, the machine learning algorithm comprises multivariate analysis carried out on a computer to arrive at a predictive model with weightage coefficients for each of the assays that minimizes the deviation between the predicted clinical response and the observed clinical response for the number of individuals (i.e., maximizes the correlation between output (e.g., sensitivity index) and clinical outcome for the number of individuals). In some embodiments, the sensitivity indices have a positive predictive value (PPV) greater than at least about 80% (such as greater than at least about 81, 82, 83, 84, 85, 86, 87, 88, 89, 90, 91, 92, 93, 94, 95, 96, 97, 98, or 99%). In some embodiments, the sensitivity indices have a negative predictive value (NPV) greater than at least about 80% (such as greater than at least about 81, 82, 83, 84, 85, 86, 87, 88, 89, 90, 91, 92, 93, 94, 95, 96, 97, 98, or 99%). In some embodiments, clinical outcomes for the number of individuals are assessed after completion of at least 3 (such as at least 3, 4, 5, 6, or more) cycles of treatment. In some embodiments, the number of individuals is at least about 50 (such as at least about any of 60, 70, 80, 90, 100, 200, 300, 400, 500, 600, 700, 800, 900, 1000, 1250, 1500, 1750, 2000, or more, including any ranges between these values).

In some embodiments, the methods described herein employ a machine learning algorithm trained on a training set. In some embodiments, the training set comprises n examples (xᵢ,yᵢ), i=1, . . . , n, wherein xᵢ is a feature vector comprising m assessment scores for the i-th patient and yᵢ is a value corresponding to clinical response for the i-th patient (e.g., 1 if the i-th patient is a responder and −1 if the i-th patient is a non-responder). In some embodiments, the machine learning algorithm is trained on the training set such that the false positive rate is less than about 30% (such less than about any of 25, 20, 15, 10, 9, 8, 7, 6, 5, 4, 3, 2, or 1%). In some embodiments, the machine learning algorithm is trained on the training set in one stage. For example, in some embodiments, the machine learning algorithm is trained on the training set in one stage to predict response or non-response for new test cases. In some embodiments, the machine learning algorithm is trained on the training set in one stage to predict response or non-response for new test cases, wherein yᵢ is 1 if the i-th patient is a responder and −1 if the i-th patient is a non-responder. In some embodiments, the machine learning algorithm is trained on the training set in at least 2 (such as at least 3, 4, 5, or more) stages. For example, in some embodiments, the machine learning algorithm is trained on the training set in at least 2 (such as at least 3, 4, 5, or more) stages to predict non-response and 2 or more classes of response (e.g., complete response and partial response) for new test cases. For example, in some embodiments, the machine learning algorithm is trained on the training set in a first stage and a second stage to predict non-response, complete response, and partial response for new test cases, wherein the first stage comprises training the machine learning algorithm on the training set to generate an initial model for response/non-response, and wherein the second stage comprises further refining the initial model to classify the predicted responders as partial-responders or complete responders.

In some embodiments, the machine learning algorithm is the SVMpAUC algorithm (Narasimhan, N. & Agarwal, S. Proceedings of the 19th ACM SIGKDD Conference on Knowledge Discovery and Data Mining. 167-175, 2013). In some embodiments, the SVMpAUC algorithm is trained on a training set comprising n examples (xᵢ,yᵢ), i=1, . . . , n, wherein xᵢ is a feature vector containing m assessment scores for the i-th patient and yᵢ is 1 if the i-th patient is a responder and −1 otherwise. In some embodiments, the SVMpAUC algorithm learns a model comprising a weight vector w comprising weightage coefficients for each of the m assessment scores maximizing (a concave lower bound on) the partial area under the ROC curve (partial AUC) up to a specified false positive rate β (e.g., β=0.25), defined as

$$pAUC ( w ) = {\sum }_{i \text{:} {y}_{i} = 1} {\sum }_{j \text{:} {y}_{j} = - 1} 1 ( w \cdot {x}_{i} > w \cdot {x}_{j} ) \cdot 1 ( j ∈ {S}_{β} ) ,$$

wherein S_{β} contains indices j of the top β fraction of non-responders in the training set, ranked according to scores w.xⱼ (Chu, W. & Keerthi, S. S. Neural Comput. 19, 792-815, 2007). In some embodiments, the model further comprises a first threshold value separating non-responders from responders in the training set with a false positive rate of about β. In some embodiments, the model further comprises a second threshold value separating partial responders from complete responders, wherein the second threshold value is selected to maximize the classification accuracy of the model for partial responders and complete responders on the training set.

In some embodiments, the possible numeric assessment scores and associated weightage coefficients for each of the assays included in the output (e.g., sensitivity index) generation for a predictive model are selected such that the output (e.g., sensitivity index) can range from a predetermined minimum to a predetermined maximum. In some embodiments, the minimum is 0 and the maximum is 100. In some embodiments, the output (e.g., sensitivity index) predicts varying degrees of responsiveness to one or more therapeutic agents in the individual. In some embodiments, the output (e.g., sensitivity index) predicts at least 2 (such as at least 2, 3, 4, 5, 6, or more) degrees of responsiveness to one or more therapeutic agents in the individual. In some embodiments, the output (e.g., sensitivity index) predicts clinical response or no clinical response to one or more therapeutic agents in the individual. In some embodiments, the output (e.g., sensitivity index) predicts complete clinical response, partial clinical response, or no clinical response to one or more therapeutic agents in the individual. In some embodiments, the output (e.g., sensitivity index) predicts complete clinical response, partial clinical response, no response, or no clinical response to one or more therapeutic agents in the individual. In some embodiments, the output (e.g., sensitivity index) is generated such that one or more threshold values separate ranges in the output (e.g., sensitivity index) that correlate with a degree of response to one or more therapeutic agents in the individual. In some embodiments, the output (e.g., sensitivity index) is generated such that a value above a threshold value predicts clinical response and a value below the threshold value predicts no clinical response in the individual. In some embodiments, the output (e.g., sensitivity index) is generated such that a value above an upper threshold value predicts complete clinical response, a value between the upper threshold value and a lower threshold value predicts partial clinical response, and a value below the lower threshold value predicts no clinical response in the individual. Such configurations can be adapted to accommodate prediction of any number of degrees of responsiveness. In some embodiments, the output (e.g., sensitivity index) range and the one or more threshold values are predetermined, such as to maximize ability to discriminate between degrees of clinical outcomes, and used as inputs in the machine learning algorithm for assigning weightage coefficients. For example, in some embodiments, a) the output (e.g., sensitivity index) can range from 0 to 100, and has an upper threshold value of 60 and a lower threshold value of 20; and b) the machine learning algorithm outputs weightage coefficients for each of the plurality of assays to maximize i) correlation of sensitivity indices ranging from 0-20 with no clinical response; ii) correlation of sensitivity indices ranging from 20-60 with partial clinical response; and iii) correlation of sensitivity indices ranging from 60-100 with complete clinical response. Various output (e.g., sensitivity index) ranges and numbers and values of thresholds are contemplated, and can be selected to suit any given purpose for predicting any number of degrees of responsiveness.

### EXAMPLES

The examples, which are intended to be purely exemplary of the invention and should therefore not be considered to limit the invention in any way, also describe and detail aspects and embodiments of the invention discussed above. The examples are not intended to represent that the experiments below are all or the only experiments performed.

### Example 1. Therapy-Induced Priming of Natural Killer Cells

Here, we employed a patient-derived ex-vivo tumor explant culture system based on a tumor microenvironment platform (see US Patent No. 2014/0228246), which serves to mimic the native 3D tumor microenvironment, autocrine-paracrine dynamic, and response to therapy by incorporating fresh tumor tissue and autologous immune cells.

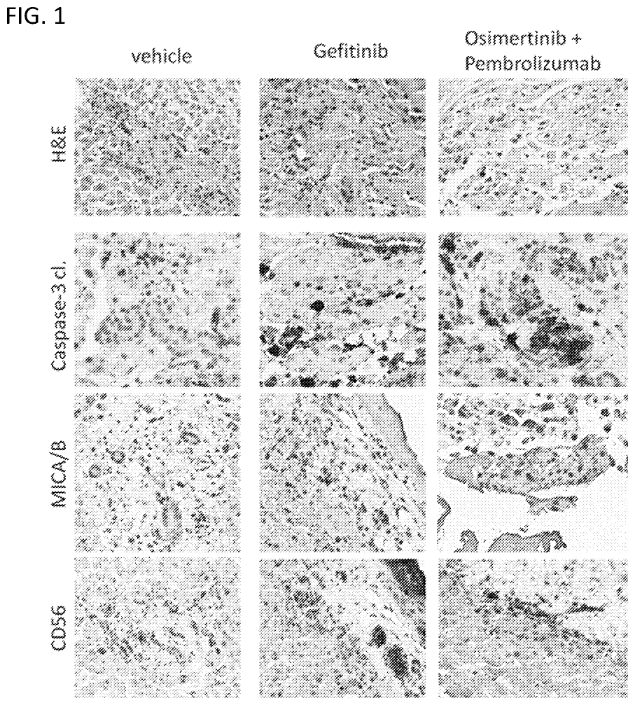

The relevance of changes in the immune contexture of the tumor tissue culture microbed for a non-immunmodulatory agent, either as monotherapy or in combination with an immunomodulatory agent, was examined. Human lung cancer sections from the same patient cultured for 72 hours in the tumor microenvironment platform with gefitinib (a small molecule kinase inhibitor) alone, osimertinib (another small molecule kinase inhibitor)+Pembrolizumab, or vehicle control were assayed for H&E staining and cleaved caspase 3, MICA/B (cell surface ligands that bind immunoreceptors present on natural killer (NK) cells), and CD56 (marker of NK cells) expression. Results are shown in FIG. 1. Cleaved caspase 3 expression was significantly increased in both treatment arms, a result that correlates with antitumor activity. Interestingly, there was also an observed increase in tumor cell MICA/B expression and infiltration of NK cells in both treatment arms vs. the vehicle-treated cohorts. Together, these data suggest that an increase in immune-reactive cells in the tumor tissue culture microbed correlates with antitumor response not only of therapeutic agents that affect the immune compartment (Pembrolizumab), but importantly also conventional non-immunomodulatory chemotherapeutic agents (Gefitinib).

Utilizing tissue from patients diagnosed with luminal, HER2 positive/negative, and triple-negative (ER− PR− HER2−) breast cancers cultured in the tumor microenvironment platform, we studied spatial heterogeneity of CD56+ lymphocytes (NK) in the tumor:stroma and phenotypic alterations under control conditions or during pressure of conventional chemotherapy and immune checkpoint blockade. The tumor tissue cultures were also evaluated using standard assays for tumor proliferation, tumor cell death, tumor morphology, and tumor cell viability as previously described, including tetrazolium salt WST-1 viability assay; LDH release; ATP uptake; glucose uptake; Caspase 3, Caspase 8, and Ki67 expression; and H&E staining. The results of the assays for tumor-associated markers were used to generate assessment scores that were input into a machine-trained algorithm to generate a clinical outcome predictor in the form of an “M-score” for each patient.

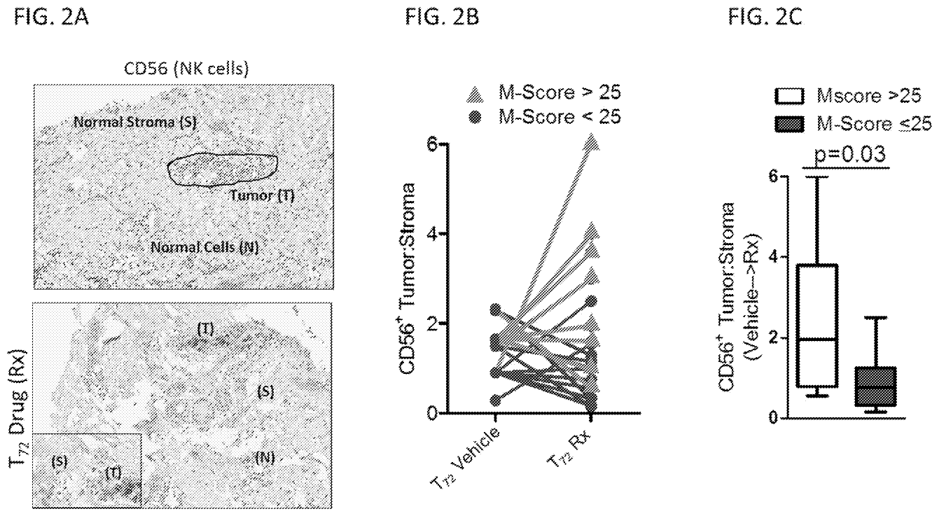

The spatial heterogeneity of CD56+ lymphocytes (NK) in areas of tumor vs stroma in pairs of tissues (for control and treatment conditions) from each of a number of patients cultured in the tumor microenvironment platform and treated with various conventional and immuno-modulatory therapies was investigated. IHC analysis for CD56+ cells in slices of tissues under treatment and control conditions was performed, and the number of CD56+ cells in tumor and stromal regions was determined (FIG. 2A). M-score based on tumor-associated markers was determined for each of the tissue pairs as previously described. The ratio of CD56+ cells in areas of tumor cells vs areas of stroma in untreated and drug pressure conditions from the tissue pairs was calculated (FIG. 2B), and the fold change from vehicle to treatment conditions for each pair was determined and binned by M-score >25 and M-score ≤25 (FIG. 2C).

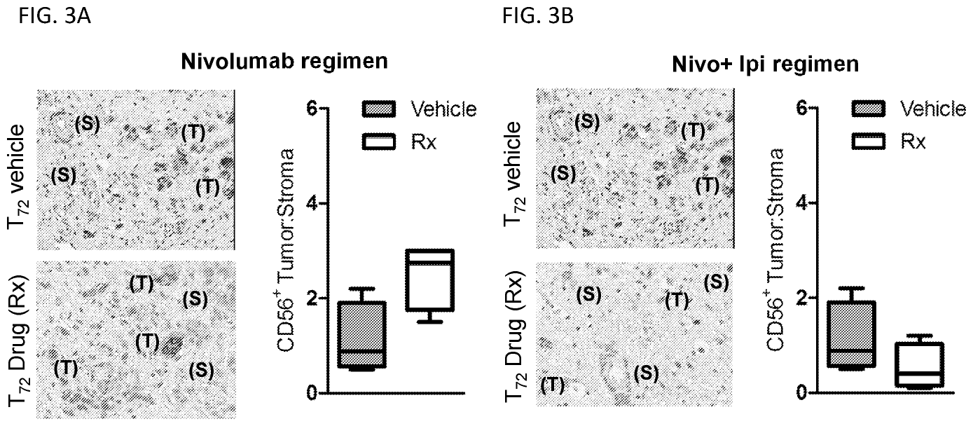

The spatial heterogeneity of CD56+NK cells in areas of tumor vs stroma in tissue from a single metastatic breast cancer patient cultured in the tumor microenvironment platform under control conditions or under pressure from treatment with immunotherapy-containing drug regimens nivolumab+adriamycin (“Nivolumab regimen,” FIG. 3A) and gemcitabine+nivolumab+ipilimumab (“Nivo+Ipi regmin,” FIG. 3B) was investigated. The number of CD56+ cells in tumor and stromal regions under treatment and control conditions was determined, and the tumor:stroma ratio of CD56+ cells was calculated (FIGS. 3A and 3B). M-score based on tumor-associated markers for the patient was determined as previously described. Treatment with nivolumab+adriamycin resulted in an M-score of 29 (predicted to respond), and was associated with an increase in the tumor:stroma ratio of NK cells. Treatment with gemcitabine+nivolumab+ipilimumab resulted in an M-score of 10 (predicted to not respond), and was associated with a decrease in the tumor:stroma ratio of NK cells.

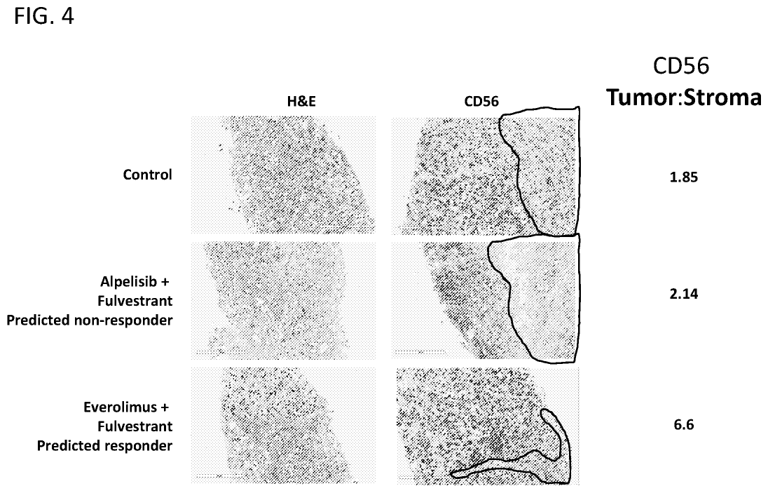

The spatial heterogeneity of CD56+NK cells in areas of tumor vs stroma in tissue from a single breast adenocarcinoma patient cultured in the tumor microenvironment platform under control conditions or under pressure from treatment with anticancer drug regimens alpelisib+fulvestrant and everolimus+fulvestrant (FIG. 4) was investigated. The number of CD56+ cells in tumor and stromal regions under treatment and control conditions was determined, and the tumor:stroma ratio of CD56+ cells was calculated (FIG. 4). M-score based on tumor-associated markers for the patient was determined as previously described. Treatment with alpelisib+fulvestrant resulted in an M-score predicting no response, and was associated with a slight increase in the tumor:stroma ratio of NK cells. Treatment with everolimus+fulvestrant resulted in an M-score predicting response, and was associated with a much greater increase in the tumor:stroma ratio of NK cells.

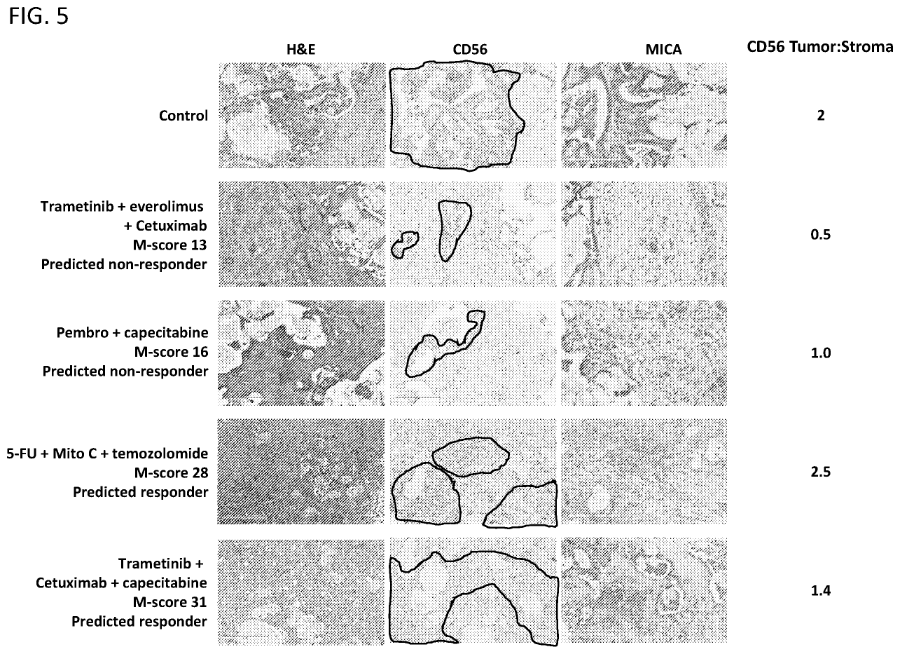

The spatial heterogeneity of CD56+NK cells in areas of tumor vs stroma in tissue from a single colon adenocarcinoma patient cultured in the tumor microenvironment platform under control conditions or under pressure from treatment with anticancer drug regimens i) trametinib+everolimus+cetuximab, ii) pembrolizumab+capecitabine, iii) 5-FU+mitomycin C+temezolomide, and iv) trametinib+cetuximab+capecitabine was investigated (FIG. 5). The number of CD56+ cells in tumor and stromal regions under treatment and control conditions was determined, and the tumor:stroma ratio of CD56+ cells was calculated (FIG. 5). M-score based on tumor-associated markers for the patient was determined as previously described. Treatment with i) trametinib+everolimus+cetuximab and ii) pembrolizumab+capecitabine resulted in M-scores predicting no response, and each was associated with a decrease in the tumor:stroma ratio of NK cells. Treatment with iii) 5-FU+mitomycin C+temezolomide and iv) trametinib+cetuximab+capecitabine resulted in M-scores predicting response, and was associated with an increase (5-FU+mitomycin C+temezolomide) or slight decrease (trametinib+cetuximab+capecitabine) in the tumor:stroma ratio of NK cells.

The predictive power of the change in spatial heterogeneity of CD56+NK cells in areas of tumor vs stroma under pressure from treatment with anticancer drug regimens was evaluated. The M-score based on tumor-associated markers and tumor:stroma ratio of CD56+ cells in tumor tissue cultures under control conditions or under pressure from treatment with anticancer drug regimens was determined as described above for 3 different patients whose clinical outcome in response to the respective treatment was known. As shown in Table 1, each of the patients incorrectly predicted to not respond based on M-score (patients 2 and 3) showed an increase in the tumor:stroma ratio of NK cells. These results suggest that incorporating the change in tumor:stroma ratio of NK cells under treatment pressure in a predictive model of clinical response based on tumor-associated markers can improve the prediction accuracy of such a model.

TABLE 1

| | Patient | | |
|---|---|---|---|
| | 1 | 2 | 3 |
| Cancer type | gastrointestinal | metastatic | metastatic |
| | stromal tumor | pancreatic cancer | pancreatic cancer |
| Drug regimen | topotecan | FOLFIRINOX | FOLFIRINOX |
| M-score | Responder | Non-responder | Non-responder |
| prediction | (M-score = 36) | | |
| Control CD56+ | 3 | 4 | 2 |
| tumor:stroma | | | |
| ratio | | | |
| Treatment CD56+ | 5 | 9 | 3 |
| tumor:stroma | | | |
| ratio | | | |
| Clinical response | Responder | Responder | Responder |

Changes in the cytokine profile (e.g., anti-inflammatory and pro-inflammatory cytokines) following therapy pressure were also investigated.

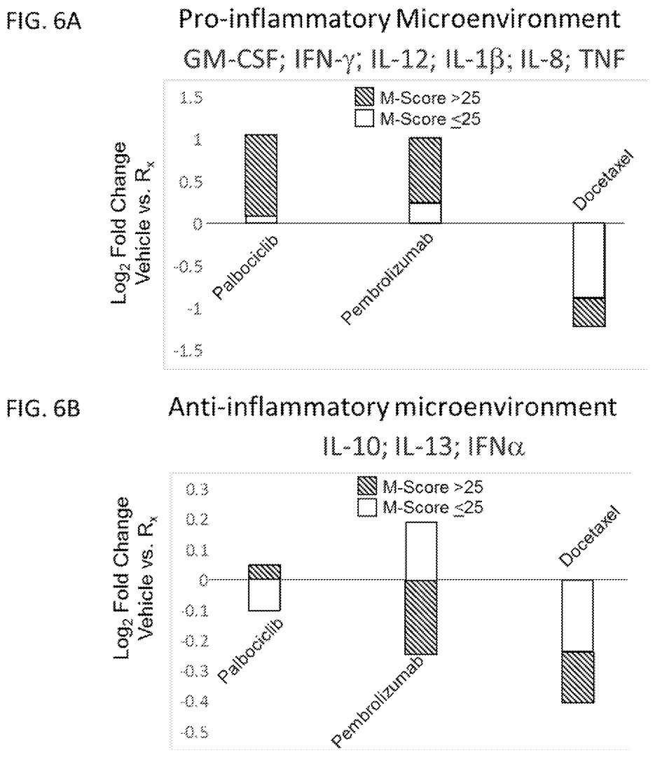

The cytokine profile in pairs of treated and untreated tumor tissue cultures from each of a number of HER2-/ER+/PR+ breast cancer patients cultured in the tumor microenvironment platform and treated with conventional chemotherapies (palbociclib or docetaxel) or immune checkpoint blockade (pembrolizumab) was investigated. The expression level of pro-inflammatory cytokines GM-CSF, IFN-γ, IL-12, IL-1β, IL-8, and TNF and anti-inflammatory cytokines IL-10, IL-13, and IFNα was determined by quantitative luminex cytokine array for the treated and untreated tumor tissue cultures. For each pair of patient-derived tumor tissue cultures, the expression levels of all of the pro-inflammatory cytokines were averaged independently for the treated and untreated tumor tissue cultures to generate treated and untreated pro-inflammatory cytokine averages for each patient, and the expression levels of all of the anti-inflammatory cytokines were averaged independently for the treated and untreated tumor tissue cultures to generate treated and untreated anti-inflammatory cytokine averages for each patient. These values were then divided (treatment/vehicle) to determine the fold change from vehicle to treatment. M-score based on tumor-associated markers for each patient was determined as previously described. The data were binned by M-score >25 and M-score ≤25, and the average fold change from untreated to treated conditions for the pro- and anti-inflammatory cytokine averages in each bin was determined and is depicted in FIGS. 6A and 6B.

Taken together, these data demonstrate a role for NK cells in the antitumor effect of cancer therapy, including conventional and immuno-modulatory anticancer drugs.

### Exemplary Embodiments

### Embodiment 1

A method of predicting responsiveness of an individual having a cancer to administration of an anticancer drug regimen, the method comprising:

a) obtaining a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell;
b) inputting the readout into a predictive model;
c) using the predictive model to generate an output; and
d) using the output to predict responsiveness of the individual to administration of the anticancer drug regimen.

### Embodiment 2

A method of classifying likely responsiveness of an individual having a cancer to administration of an anticancer drug regimen, comprising:

a) obtaining a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell;
b) inputting the readout into a predictive model;
c) using the predictive model to generate an output; and
d) using the output to classify the likely responsiveness of the individual to administration of the anticancer drug regimen.

### Embodiment 3

A computer-implemented method for predicting responsiveness of an individual having a cancer to administration of an anticancer drug regimen, the method comprising:

a) accessing a readout comprising an assessment score for each of a plurality of assays conducted on a tumor tissue culture treated with the anticancer drug regimen, wherein the tumor tissue culture comprises a tumor tissue from the individual cultured on a tumor microenvironment platform, wherein the plurality of assays comprises a first set of a plurality of assays and a second set of one or more assays, and wherein the second set of one or more assays comprises an assay for tumor infiltration of an immune cell;
b) inputting the readout into a predictive model;
c) using the predictive model to generate an output; and
d) using the output to predict responsiveness of the individual to administration of the anticancer drug regimen.

### Embodiment 4

The method of any one of embodiments 1-3, wherein the predictive model comprises a first algorithm that uses each of the assessment scores as input and generates the output.

### Embodiment 5

The method of embodiment 4, wherein the first algorithm comprises multiplying each of the input assessment scores with a corresponding weightage coefficient to obtain a plurality of weighted assessment scores; and combining the plurality of weighted assessment scores to generate the output.

### Embodiment 6

The method of any one of embodiments 1-3, wherein the predictive model comprises a first algorithm that uses each of the assessment scores for the first set of a plurality of assays as input and generates a preliminary output, and a second algorithm that uses the preliminary output and each of the assessment scores for the second set of one or more assays as input and generates the output.

### Embodiment 7

The method of embodiment 6, wherein the first algorithm comprises multiplying each of the input assessment scores with a corresponding weightage coefficient to obtain a plurality of weighted assessment scores; and combining the plurality of weighted assessment scores to generate the preliminary output.

### Embodiment 8

The method of embodiment 6 or 7, wherein the preliminary output predicts a primary degree of responsiveness of the individual to administration of the anticancer drug regimen, and wherein the second algorithm comprises adjusting the primary predicted degree of responsiveness based on the input assessment scores to generate the output.

### Embodiment 9

The method of embodiment 8, wherein the second set of one or more assays consists of the assay for tumor infiltration of an immune cell, and wherein adjusting the primary predicted degree of responsiveness comprises predicting a secondary degree of responsiveness of the individual to administration of the anticancer drug regimen based on the input assessment score, and

1) adjusting the primary predicted degree of responsiveness by decreasing the predicted degree of responsiveness if the secondary predicted degree of responsiveness is lower than the primary predicted degree of responsiveness and the input assessment score is below a first threshold, thereby generating the output; or
2) adjusting the primary predicted degree of responsiveness by increasing the predicted degree of responsiveness if the secondary predicted degree of responsiveness is greater than the primary predicted degree of responsiveness and the input assessment score is above a second threshold, thereby generating the output.

### Embodiment 10

The method of any one of embodiments 1-9, wherein the output predicts complete clinical response, partial clinical response, or no clinical response of the individual to administration of the anticancer drug regimen.

### Embodiment 11

The method of any one of embodiments 1-9, wherein the output predicts response or no response of the individual to administration of the anticancer drug regimen.

### Embodiment 12

The method of any one of embodiments 1-11, wherein the assay for tumor infiltration of an immune cell comprises determining the amount of the immune cell in a region of tumor cells in the tumor tissue culture.

### Embodiment 13

The method of embodiment 12, wherein the assay for tumor infiltration of an immune cell comprises determining the ratio of i) the amount of the immune cell in a region of tumor cells in the tumor tissue culture to ii) the amount of the immune cell in a region of normal stroma in the tumor tissue culture.

### Embodiment 14

The method of any one of embodiments 1-13, wherein the immune cell is an NK cell.

### Embodiment 15

The method of any one of embodiments 1-14, wherein the first set of a plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof.

### Embodiment 16

The method of any one of embodiments 1-15, wherein the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C.

### Embodiment 17

The method of embodiment 16, wherein the tumor microenvironment platform further comprises serum, plasma, and/or peripheral blood nuclear cells (PBNCs).

### Embodiment 18

The method of embodiment 17, wherein one or more of the serum, plasma, and/or PBNCs are derived from the individual.

### Embodiment 19

The method of any one of embodiments 1-18, wherein step a) further comprises conducting the plurality of assays on the tumor tissue culture and/or step a) further comprises preparing the tumor tissue culture by culturing tumor tissue from the individual on the tumor microenvironment platform.

### Embodiment 20

The method of any one of embodiments 1-19, wherein the assessment scores are generated based on a comparison between i) the results of the plurality of assays conducted on the tumor tissue culture treated with the anticancer drug regimen; and ii) the results of the plurality of assays conducted on a reference tumor tissue culture, wherein the reference tumor tissue culture comprises a tumor tissue from the individual cultured on the tumor microenvironment platform.

### Embodiment 21

The method of embodiment 20, wherein the reference tumor tissue culture is not treated with the anticancer drug regimen.

### Embodiment 22

The method of embodiment 20 or 21, wherein step a) further comprises conducting the plurality of assays on the reference tumor tissue culture; and/or step a) further comprises preparing the reference tumor tissue culture by culturing tumor tissue from the individual on the tumor microenvironment platform.

### Embodiment 23

A method of treating cancer in an individual in need thereof, the method comprising administering to the individual an anticancer drug regimen to which the individual is predicted to respond according to the method of any one of embodiments 1-22.

### Embodiment 24

The method of embodiment 23, wherein the individual is predicted to have a complete clinical response or partial clinical response to administration of the anticancer drug regimen.

### Embodiment 25

The method of any one of embodiments 1-24, wherein the anticancer drug regimen comprises an anticancer agent and/or an immunotherapeutic agent.

### Embodiment 26

The method of embodiment 25, wherein the anticancer agent is selected from the group consisting of adriamycin, gemcitabine, palbociclib, docetaxel, fulvestrant, carboplatin, exemestane, everolimus, vinorelbine, olaparib, capecitabine, cyclophosphamide, methotrexate, fluorouracil, and any combination thereof.

### Embodiment 27

The method of embodiment 25 or 26, wherein the immunotherapeutic agent is an immune checkpoint inhibitor.

### Embodiment 28

The method of embodiment 25 or 26, wherein the immunotherapeutic agent is selected from the group consisting of nivolumab, ipilimumab, pembrolizumab, atezolizumab, and any combination thereof.

### Embodiment 29

The method of any one of embodiments 1-28, wherein the individual is human.

1.-29. (canceled)

30. A method comprising:
a) culturing a tumor tissue obtained from an individual on a tumor microenvironment platform to obtain a tumor tissue culture,
wherein the tumor microenvironment platform comprises peripheral blood nuclear cells;
b) conducting an assay on the tumor tissue culture treated with an anticancer drug regimen;
wherein the assay comprises determining i) an amount of an immune cell in a region of tumor cells in the tumor tissue culture and ii) an amount of the immune cell in a region of normal stroma in the tumor tissue culture.

31. The method of claim 30, further comprising the step of generating an assessment score for the assay.

32. The method of claim 31, further comprising the step of using the assessment score to predict responsiveness of the individual to administration of the anticancer drug regimen.

33. The method of claim 30, wherein said determining in step (b) comprises determining a ratio of i) an amount of an immune cell in a region of tumor cells in the tumor tissue culture to ii) an amount of the immune cell in a region of normal stroma in the tumor tissue culture.

34. The method of claim 30, wherein the immune cell is a natural killer cell.

35. The method of claim 30, wherein the immune cell is a CD56+ cell.

36. The method of claim 30, further comprising:
a) conducting one or more additional assays on the tumor tissue culture treated with the anticancer drug regimen,
wherein the one or more additional assays is selected from a group consisting of a cell viability assay, a cell death assay, a cell proliferation assay, a tumor morphology assay, a tumor stroma content assay, a cell metabolism assay, a senescence assay, a cytokine profile assay, an enzyme activity assay, a tumor:stromal cell expression, and any combination thereof; and
b) generating an assessment score for each assay in the one or more additional assays.

37. The method of claim 36, further comprising:
a) multiplying each assessment score with a corresponding weightage coefficient to obtain a plurality of weighted assessment scores;
b) combining the plurality of weighted assessment scores to generate an output; and
c) using the output to predict responsiveness of the individual to administration of the anticancer drug regimen.

38. The method of claim 30, wherein the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C.

39. The method of claim 30, wherein the anticancer drug regimen is selected from a group consisting of a chemotherapeutic agent, a targeted therapeutic agent, an immunotherapeutic agent, and any combination thereof.

40. The method of claim 30, wherein the peripheral blood nuclear cells are derived from said individual.

41. A method of predicting responsiveness of an individual having a cancer to administration of an anticancer drug regimen, the method comprising:
a) obtaining a readout comprising an assessment score for each of a plurality of assays conducted on a tumor tissue culture treated with the anticancer drug regimen,
wherein the tumor tissue culture comprises a tumor tissue from the individual cultured on a tumor microenvironment platform, and
wherein the plurality of assays comprises a first set of a plurality of assays and a second set of one or more assays, wherein the second set of one or more assays comprises determining the ratio of i) an amount of an immune cell in a region of tumor cells in the tumor tissue culture to ii) an amount of the immune cell in a region of normal stroma in the tumor tissue culture;
b) inputting the readout into a predictive model;
c) using the predictive model to generate an output; and
d) using the output to predict responsiveness of the individual to administration of the anticancer drug regimen.

42. The method of claim 41, wherein the predictive model comprises a first algorithm that uses each of the assessment scores as input and generates the output.

43. The method of claim 42, wherein the first algorithm comprises multiplying each of the assessment scores with a corresponding weightage coefficient to obtain a plurality of weighted assessment scores; and combining the plurality of weighted assessment scores to generate the output.

44. The method of any one of claim 41, wherein the predictive model comprises i) a first algorithm that uses each of the assessment scores for the first set of the plurality of assays as input and generates a preliminary output, and ii) a second algorithm that uses the preliminary output and each of the assessment scores for the second set of one or more assays as input and generates the output.

45. The method of claim 44, wherein the first algorithm comprises i) multiplying each of the assessment scores for the first set of the plurality of assays with a corresponding weightage coefficient to obtain a plurality of weighted assessment scores; and ii) combining the plurality of weighted assessment scores to generate the preliminary output.

46. The method of claim 45, wherein the preliminary output predicts a primary degree of responsiveness of the individual to administration of the anticancer drug regimen, and wherein the second algorithm comprises adjusting the primary degree of responsiveness based on the assessment scores from the second set of one or more assays to generate the output.

47. The method of claim 46, wherein adjusting the primary degree of responsiveness comprises:
i) decreasing the primary degree of responsiveness if the assessment scores from the second set of one or more assays is below a first threshold, thereby generating the output; or
ii) increasing the primary degree of responsiveness if the assessment scores from the second set of one or more assays is above a second threshold, thereby generating the output.

48. The method of claim 47, wherein the output predicts clinical response or no response of the individual to administration of the anticancer drug regimen.

49. The method of claim 47, wherein the output predicts complete clinical response, partial clinical response, or no clinical response of the individual to administration of the anticancer drug regimen.

50. The method of claim 41, wherein the immune cell is a natural killer cell.

51. The method of claim 41, wherein the immune cell is a CD56+ cell.

52. The method of claim 41, wherein the first set of a plurality of assays is selected from a group consisting of a cell viability assay, a cell death assay, a cell proliferation assay, a tumor morphology assay, a tumor stroma content assay, a cell metabolism assay, a senescence assay, a cytokine profile assay, an enzyme activity assay, a tumor:stromal cell expression assay, and any combination thereof.

53. The method of claim 41, wherein the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C.

54. The method of claim 53, wherein the tumor microenvironment platform further comprises one or more of serum, plasma, and peripheral blood nuclear cells (PBNCs).

55. The method of claim 54, wherein one or more of the serum, plasma, and PBNCs are derived from the individual.

56. The method of claim 41, wherein the assessment scores are generated based on a comparison between i) the results of the plurality of assays conducted on the tumor tissue culture treated with the anticancer drug regimen; and ii) the results of the plurality of assays conducted on a reference tumor tissue culture,
wherein the reference tumor tissue culture comprises a tumor tissue from the individual cultured on the tumor microenvironment platform, and wherein the reference tumor tissue culture is not treated with the anticancer drug regimen.

57. A method of treating an individual, the method comprising administering to the individual the anticancer drug regimen if the individual is predicted to respond to the anticancer drug regimen, according to the method of claim 41.

58. The method of claim 41, wherein the anticancer drug regimen is selected from a group consisting of a chemotherapeutic agent, a targeted therapeutic agent, an immunotherapeutic agent, and any combination thereof.
